(19) United States
(12) Patent Application Publication
(10) Pub. No.: US 2024/0264141 A1
(43) Pub. Date: Aug. 8, 2024

(54) METHOD AND DEVICE FOR CELL DISCRIMINATION USING ARTIFICIAL INTELLIGENCE

(71) Applicant: Korea University Research and Business Foundation, Seoul (KR)

(72) Inventors: Sung-Hoi HONG, Seoul (KR); Min-Jae KIM, Seoul (KR)

(73) Assignee: Korea University Research and Business Foundation, Seoul (KR)

(21) Appl. No.: 18/563,869

(22) PCT Filed: Oct. 21, 2022
(86) PCT No.: PCT/KR2022/016153

§ 371 Date: Nov. 22, 2023

(30) Foreign Application Priority Data
Oct. 25, 2021 (KR) 10-2021-0142481
Oct. 20, 2022 (KR) 10- 2022-0135609

(51) Int. Cl. G01N 33/483; G06T 7/00

(52) U.S. Cl. CPC G01N 33/4833; G06T 7/0016; G06T 2207/20081; G06T 2207/20084; G06T 2207/30024

(57) ABSTRACT

The present invention relates to a method and device for cell discrimination, using artificial intelligence and, in the present invention, when various types of cells are cultured in various media, the fine morphology of the initially changing cells may be learned to observe only cell images, thus distinguishing and determining the unique characteristics of the cells. The method for cell discrimination using artificial intelligence according to the present invention may include an inputting step of inputting cell images and a discriminating step of discriminating at least one of various cell types, various culturing conditions, and various culturing times corresponding to the input cell image using a deep learning-based discriminating model.

### FIELD

The present disclosure relates to a cell discriminating method and device using artificial intelligence. More specifically, the present disclosure relates to a cell analysis method and device using artificial intelligence in which when culturing various types of cells in various types of media, the fine morphology of the cells initially changing is learned and only the cell image is observed based on the morphology, and the unique characteristics of the cells are distinguished from each other and are determined based on the images.

### DESCRIPTION OF RELATED ART

Cell culturing is an important technology in biological research, including molecular biology, and means culturing specific cells for purposes such as diagnosis or treatment of human diseases. Recently, efficient mass culturing of cells and tissues (collectively referred to as “cells”) has been required in fields such as pharmaceutical production, gene therapy, regenerative medicine, and immunotherapy. In particular, stem cells are the most actively researched topic in the bio-life field. The stem cells are differentiated into cells with specific functions depending on the environment and stimulation. Thus, in order to discover action mechanisms or induction methods thereof, it is essential to analyze and track the cell differentiation process during culturing thereof.

During cell culturing and differentiation experiments, researchers must identify whether the cells are being cultivated or differentiated to their original desired shapes or characteristics. It is almost impossible to perfectly detect minute cellular changes with the human eyes. This also limits the ability to determine the success of a differentiation experiment during the cell differentiation process.

Accordingly, as a result of the diligent efforts for the present inventors to determine the characteristics of cells only based on cell images without additional equipment, we have identified that when analyzing minute cellular changes using a convolution neural network (hereinafter, referred to as CNN) of artificial intelligence deep learning, the cell characteristics may be analyzed at high accuracy. In this way, we have completed the present disclosure.

The above information in this Background section is intended solely to improve understanding of the background of the present disclosure. Accordingly, the above information may not include information that is admitted as the prior art already known to those with ordinary knowledge in the technical field to which the present disclosure belongs. (Patent Document 1) KR 10-2084683 B1 (Cell image analysis method and cell image processing device using artificial neural network)

### DISCLOSURE

### Technical Purpose

Therefore, the present disclosure has been designed to solve the above problem. Thus, a purpose of the present disclosure is to provide a cell discriminating method and device using artificial intelligence deep learning in which cell images that change across various time zones in culturing various types of cells including stem cells under various culturing conditions are obtained, and are pre-learned, and pre-stored and managed using a deep learning-based convolution neural network, and cell characteristics in various time zones in a process of maintaining and cultivating cells and inducing cell differentiation may be distinguished from each other and may be determined based on the deep learning result.

Other purposes and advantages of the present disclosure will be described below and will be apparent based on the embodiment of the present disclosure. Furthermore, the purposes and advantages of the present disclosure may be realized by means of the means and combinations indicated in the claims.

### Technical Solution

In order to achieve the above purpose, a cell discrimination method using artificial intelligence according to an embodiment of the present invention comprise an input step of inputting a cell image; and a discriminating step of discriminating at least one of various cell types, various culturing conditions, and various culturing times corresponding to the input cell image using a deep learning-based discriminating model, wherein the discriminating step includes: extracting a first feature in the cell image; extracting a second feature in the cell image; and determining the at least one of the cell types, the culturing conditions, and the culturing times corresponding to the cell image, based on the extracted first feature and second feature, wherein the discriminating model includes: a first neural network configured to extract the first feature from the cell image; a second neural network configured to extract the second feature from the cell image; and a fully connected layer configured to determine the at least one of the cell types, the culturing conditions, and the culturing times corresponding to the input cell image, based on the extracted first feature and second feature.

In the cell discrimination method using artificial intelligence according to an embodiment of the present invention, wherein the cell image is captured in at least one of following time zones: 1 hour to 1 hour and 30 minutes, 3 hours to 3 hours and 30 minutes, 6 hours to 6 hours and 30 minutes, 12 hours to 12 hours and 30 minutes, and 24 hours to 24 hours and 30 minutes after the cell culturing.

In the cell discrimination method using artificial intelligence according to an embodiment of the present invention, wherein the first neural network is implemented as a shallow-structured convolution neural network composed of one convolution layer and one pooling layer, wherein the second neural network is implemented as a deep-structured convolution neural network composed of four convolution layers.

In the cell discrimination method using artificial intelligence according to an embodiment of the present invention, wherein the various cell types include animal cells and human cells including at least one of stem cell lines, human skin fibroblast cell lines, epithelial cell lines, and immune cell lines, wherein the various culturing conditions are different from each other for each cell type.

In the cell discrimination method using artificial intelligence according to an embodiment of the present invention, wherein the stem cell line includes at least one of mouse embryonic stem cell, mouse induced pluripotent stem cell, human embryonic stem cell, human induced pluripotent stem cell, human neural stem cell, human hair follicle stem cell, human mesenchymal stem cell, and human fibroblast cell, wherein the epithelial cell line includes human skin keratinocyte (HaCaT), wherein the immune cell line includes a T cell, wherein the human neural stem cells includes a human somatic cell-derived cell converted neural stem cell or a human brain-derived neural stem cell.

In the cell discrimination method using artificial intelligence according to an embodiment of the present invention, wherein the culturing condition for the mouse embryonic stem cell includes at least one of: a culturing condition including LIF (leukaemia inhibitory factor) media; a culturing condition including ITS (insulin-transferrin-selenium supplement) media; and a culturing condition excluding the LIF media, wherein the culturing condition for the mouse induced pluripotent stem cell includes at least one of: a culturing condition including PD0325901, SB431542, thiazovivin, ascorbic acid, and LIF media; a culturing condition excluding PD0325901, SB431542, thiazovivin, ascorbic acid, and LIF media; and a culturing condition including ITS media, wherein the culturing condition for the human embryonic stem cell or the human induced pluripotent stem cell includes at least one of: a culturing condition including PD0325901, SB431542, thiazovivin, ascorbic acid, and LIF media; a culturing condition excluding PD0325901, SB431542, thiazovivin, ascorbic acid, and LIF media; and a culturing condition including ITS media, wherein the culturing condition for the human somatic cell-derived cell-converted neural stem cell includes at least one of: a culturing condition including DMEM/F12, N2, B27, bFGFF, EGF, thiazovivin, valproic acid, Purmorphamine, A8301, SB431542, CHIR99021, Deazaneplanocin A (DZNep), and Azacitidine (5-AZA); a culturing condition including DMEM/F12, N2, B27, bFGF, and EGF; and a culturing condition including DMEM/F12 and ITS media, wherein the culturing condition for the human brain-derived neural stem cell includes at least one of: a culturing condition including a basic medium, an induced neural stem cell growth supplement and antibiotics; a culturing condition including the basic medium and antibiotics; and a culturing condition including the basic medium, antibiotics, and ITS media, wherein the culturing condition for the human hair follicle stem cells includes at least one of: a culturing condition in which 10% FBS (Fetal bovine serum), Pen/Strep (Penicillin & Streptomycin), L-glutamine, and streptomycin are contained in DMEM media; and a culturing condition in which ITS media is contained in DMEM media, wherein the culturing condition for the human mesenchymal stem cells includes at least one of: a culturing condition in which 10% FBS (Fetal bovine serum), NEAA (non-Essential Amino Acids), and Pen/Strep are contained in DMEM media; and a culturing condition in which the ITS media is contained in DMEM media, wherein the culturing condition for the human fibroblast includes a culturing condition in which 10% FBS, Pen/Strep, and NEAA are contained in DMEM media, wherein the culturing condition for the HaCaT cells includes a culturing condition in which 10% FBS, Pen/Strep, L-glutamine, and streptomycin are contained in DMEM media.

In the cell discrimination method using artificial intelligence according to an embodiment of the present invention, wherein the discriminating model includes a data set for image learning, wherein the data set includes each of a set of 1,000 training images, a set of 1,500 training images, and a set of 2,000 training images, and a set of 800 validation images, and a set of 100 test images.

In the cell discrimination method using artificial intelligence according to an embodiment of the present invention, wherein the discriminating model is configured to adopt a set of 2,000 training images.

A cell discriminating device using artificial intelligence comprise an input unit for receiving a cell image; a discriminating unit configured to discriminate at least one of various cell types, various culturing conditions, and various culturing times corresponding to the cell image using a deep learning-based discriminating model; and an output unit configured to provide the discriminating result of the discriminating unit to a user terminal, wherein the discriminating unit is configured to: extract a first feature in the cell image; extract a second feature in the cell image; and determine the at least one of the cell types, the culturing conditions, and the culturing times corresponding to the cell image, based on the extracted first feature and second feature, wherein the discriminating model includes: a first neural network configured to extract the first feature from the cell image; a second neural network configured to extract the second feature from the cell image; and a fully connected layer configured to determine the at least one of the cell types, the culturing conditions, and the culturing times corresponding to the input cell image, based on the extracted first feature and second feature.

### Technical Effect

As described above, according to the cell discriminating method and device using artificial intelligence according to the present disclosure, the cell images that change across various time zones in culturing various types of cells including stem cells under various culturing conditions are obtained, and are pre-learned and pre-stored and managed using the deep learning-based convolution neural network, and cell characteristics across the time zones in a process of maintaining and cultivating cells and inducing cell differentiation may be distinguished from each other and may be determined based on the deep learning result.

Specifically, the present disclosure may have following effects.

First, the image of cells changing during culturing under given culturing conditions are captured on each cell type and across various time zones. Thus, the image of cells may be very useful for automatically identifying the characteristics of cells during unmanned automated cell culturing in the future, and for monitoring whether the cell culturing is performed to achieve the original desired shape or characteristics of the cells.

Second, when the researcher conducts experiments on various types of cells and under various culturing conditions in each laboratory, the CNN technology is used to detect subtle cell changes. Thus, constant and uniform cell culturing may be achieved using the detection result. Accurate cell culturing and differentiation experiments may be realized according to existing established cell culturing conditions. Thus, a new bio market related to cell culturing through constructing an unmanned automated cell culturing system in the future may be created.

Third, the Resnet50 algorithm is used to train a deep learning-based model. Thus, a model training time may be reduced, and accuracy may be increased.

### BRIEF DESCRIPTION OF THE DRAWING

FIG. 1 is a flowchart showing a cell discriminating method using artificial intelligence deep learning according to one embodiment of the present disclosure.

FIG. 2 is an example views showing cell images acquired across various time zones in the cell discriminating method using artificial intelligence deep learning according to one embodiment of the present disclosure.

FIG. 3 is an example diagram showing a structure of a discriminating model in a cell discriminating method using artificial intelligence deep learning according to one embodiment of the present disclosure.

FIG. 4 is a flowchart showing a process of applying a discriminating model to determine one or more of a cell type, a culturing condition, and culturing time in the cell discriminating method using artificial intelligence deep learning according to one embodiment of the present disclosure.

FIGS. 5 to 79 are graphs showing training results obtained by applying a discriminating model in a cell discriminating method using artificial intelligence deep learning according to one embodiment of the present disclosure, wherein FIGS. 5 to 9 show training results using a set of 1,000 training images, FIGS. 10 to 29 show training results using a set of 1,500 training images, and FIG. 30 to FIG. 79 show training results using a set of 2,000 training images.

FIG. 80 is a graph showing a comparing result of training accuracies of training sets in a cell discriminating method using artificial intelligence deep learning according to one embodiment of the present disclosure.

FIGS. 81 to 86 are graphs showing precision and recall of cell morphology learning when culturing various types of cells in various media conditions according to the present disclosure.

FIG. 87 is a block diagram showing a cell discriminating device using artificial intelligence deep learning according to one embodiment of the present disclosure.

### DETAILED DESCRIPTIONS

The terms used herein will be briefly described. A detailed description of the present disclosure will be made.

The terms used in the present disclosure are selected as widely used general terms as possible while considering a function thereof in the present disclosure. However, the meaning of the terms may vary depending on intentions of technicians working in the field, legal precedents, emergence of new schemes, etc. Furthermore, in certain cases, there are terms arbitrarily selected by the applicant, and in this case, the meaning thereof will be described in detail in the relevant description section. Therefore, the terms used in the present disclosure should be defined based on the meaning of the terms and the overall content of the present disclosure, rather than simply the name of the term.

It will be further understood that the terms “comprise”, “including”, “include”, and “including” when used herein, specify the presence of the stated components, but do not preclude the presence or addition of one or more other components. Furthermore, the terms such as “unit” and “module” used herein refer to a unit that processes at least one function or operation. The unit or the module may be implemented in a hardware or software manner, or in a combination of hardware and software. It will be understood that when an element or layer is referred to as being “connected to”, or “connected to” another element or layer, it may be directly on, connected to, or connected to the other element or layer, or one or more intervening elements or layers may be present.

Hereinafter, with reference to the attached drawings, an embodiment of the present disclosure will be described in detail so that those skilled in the art can easily implement the present disclosure. However, the present disclosure may be implemented in several different forms and is not limited to the embodiments as described herein. In addition, in order to clearly illustrate the present disclosure, parts unrelated to the description were omitted in the drawings. Similar reference numbers are assigned to similar elements throughout the present disclosure.

Hereinafter, the present disclosure will be described in detail with reference to the attached drawings.

According to one embodiment of the present disclosure, a cell discriminating method using artificial intelligence deep learning may be provided.

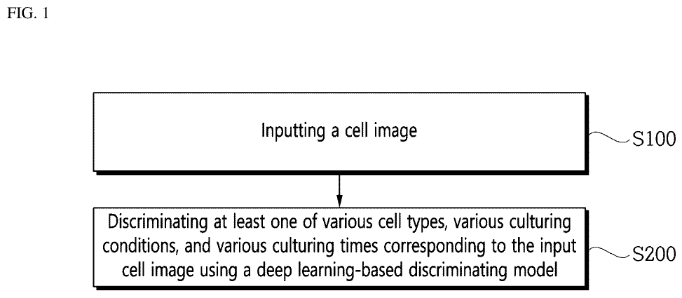

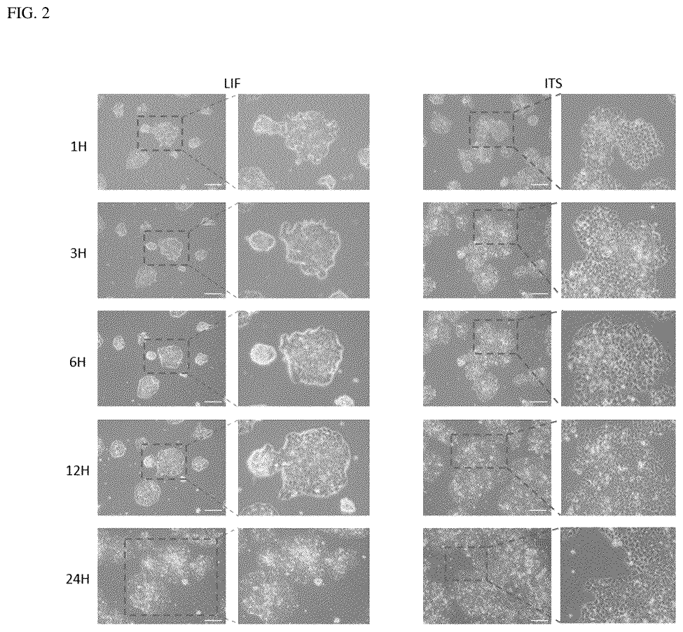

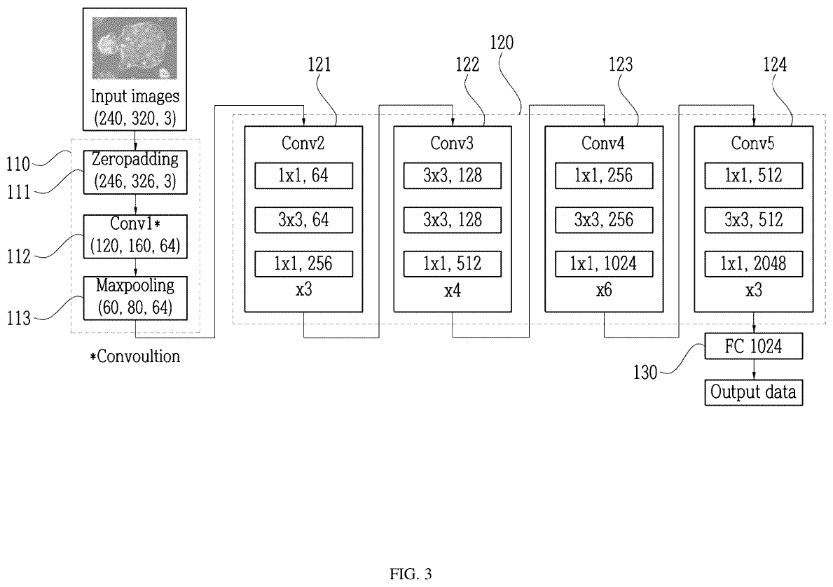

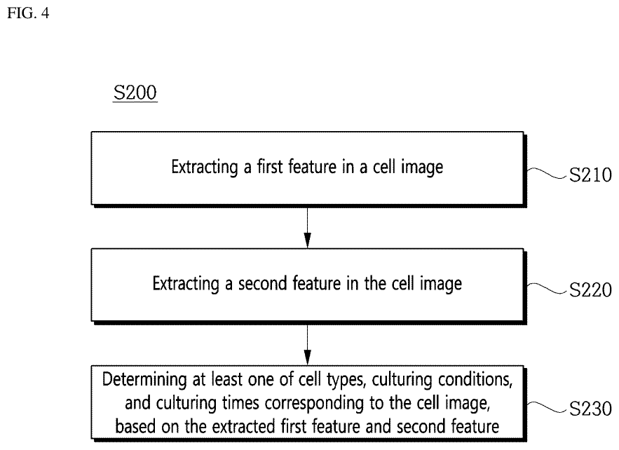

FIG. 1 is a flowchart showing a cell discriminating method using artificial intelligence deep learning according to one embodiment of the present disclosure. FIG. 2 is an example views showing cell images acquired across various time zones in the cell discriminating method using artificial intelligence deep learning according to one embodiment of the present disclosure. FIG. 3 is an example diagram showing a structure of a discriminating model in a cell discriminating method using artificial intelligence deep learning according to one embodiment of the present disclosure. FIG. 4 is a flowchart showing a process of applying a discriminating model to determine one or more of a cell type, a culturing condition, and culturing time in the cell discriminating method using artificial intelligence deep learning according to one embodiment of the present disclosure.

First, referring to FIG. 1, the cell discriminating method using artificial intelligence deep learning according to one embodiment of the present disclosure may include an input step S100 where a cell image 10 is input, and a discriminating step S200 of discriminating which of various cell types, various culturing conditions, and culturing times corresponds to the cell image 10 using a deep learning-based discriminating model 100.

In accordance with the present disclosure, the cell image 10 refers to an image of a cell acquired using an imaging device such as an optical microscope. There are no restrictions on a scheme of photographing the cell image using a photographing device.

The cell image 10 may be acquired across predetermined time zones after cell culturing.

For example, the cell image 10 may be acquired by an imaging device in any one or more of a time zone of 1 hour to 1 hour 30 minutes, a time zone of 3 hours to 3 hours 30 minutes, a time zone of 6 hours to 6 hours 30 minutes, a time zone of 12 hours to 12 hours 30 minutes, and a time zone of 24 hours to 24 hours 30 minutes after the cell culturing.

The change in the shape of the proliferating cell is greatest in the beginning of the cell culturing, and therefore though varying based on each cell line. Thus, it is best to acquire the cell images within 24 to 25 hours, preferably within 24 hours after the cell culturing.

Referring to FIG. 2, it may be identified that the cell image 10 is being acquired in a minimum time unit, that is, 1 hour, 3 hours, 6 hours, 12 hours, and 24 hours after the cell culturing.

For reference, FIG. 2 shows cell images acquired in 3 hours, 6 hours, 12 hours, and 24 hours after culturing of B6 cells among mouse embryonic stem cells (mES) in each of LIF (leukaemia inhibitory factor) media for maintenance and ITS (insulin-transferrin-selenium supplement) media for differentiation.

In accordance with the present disclosure, various culturing conditions are applied to various types of cells, including stem cells, to acquire cell images that change across various time zones. The deep learning scheme may be applied to the acquired cell images to analyze minute cell changes. Thus, the cell characteristics in each of various time zones during the process of cell maintenance and culturing or cell differentiation induction may be distinguished from each other and determined, based on the analysis result.

Various deep learning schemes may be applied to the cell discriminating method using artificial intelligence deep learning according to one embodiment of the present disclosure. In other words, a discriminating model 100 generated by learning the cell images based on the deep learning scheme may be used to discriminate which of various cell types, various culturing conditions, and culturing times corresponds to the cell image 10.

Preferably, in accordance with the present disclosure, the discriminating model 100 may be generated based on a convolution neural network (CNN) among various deep learning schemes.

More preferably, in accordance with the present disclosure, the discriminating model 100 may be generated based on a Resnet50 algorithm among various convolution neural networks (CNN).

Referring to FIG. 3, the discriminating model 100 may include a first neural network 110 configured to extract a first feature in a cell image 10, a second neural network 120 configured to extract a second feature in the cell image 10, and a fully connected layer 130 configured to determine one or more of the cell type, the culturing condition, and the culturing time corresponding to the input cell image 10, based on the extracted first feature and the second feature. In this regard, the fully connected layer may act as a classifier that determines one or more of the cell type, the culturing condition, and the culturing time corresponding to the cell image 10 based on the extracted feature information.

In this regard, the first feature of the cell image 10 may be large features, for example, a cell shape feature, and the second feature of the cell image 10 may be small features, for example, a cell edge feature.

Furthermore, the first neural network 110 may be implemented as a shallow-structured convolution neural network composed of one first convolution layer 112 (Conv1) and one pooling layer 113. The second neural network 120 may be implemented as a deep-structured convolution neural network composed of four convolution layers, that, is second to fifth convolution layers 121, 122, 123, and 124 (Conv2, Conv3, Conv4, and Conv5). In this regard, the pooling layer 113 may perform a pooling operation to reduce a size of output data of the convolution layer 112 or to emphasize specific data. The pooling layer 113 may include a max pooling layer and an average pooling layer.

Specifically, in the discriminating model 100, the cell image 10 used as the input may have a size of, for example, 240×320 pixels.

When the cell image 10 is input to the first neural network 110, the feature of the image may be extracted via the convolution layer 112 and the pooling layer 113. In this regard, since the image size is reduced after the processing, subsequent processing begins while the image size temporarily increases by performing zero padding 111 processing to maintain the image size. For example, the size of the cell image 10 is increased from 240×320 to 246×236 via the zero padding 111 processing. Accordingly, an image of 120×160×64 pixels is input to the convolution layer 112, and then an image of 60×80×64 pixels obtained via the pooling operation in the pooling layer 113 is input to the second neural network 120.

The four second to fifth convolution layers 121, 122, 123, and 124 of the second neural network 120 extract the feature of the image using a filter of 1×1 or 3×3 size.

For example, the second convolution layer 121 (Conv2) analyzes an image pattern using (1×1, 64), (3×3, 64) and (1×1, 256) filters and repeats this analysis three times. That is, the second convolution layer 121 (Conv2) is composed of 9 layers.

The third convolution layer 122 (Conv3) analyzes the image pattern via (1×1, 128), (3×3, 128) and (1×1, 512) filters, and repeats this analysis four times. That is, the third convolution layer 122 (Conv3) is composed of 12 layers.

The fourth convolution layer 123 (Conv4) analyzes the image pattern via (1×1, 256), (3×3, 256) and (1×1, 1024) filters, and repeats this analysis six times. That is, the fourth convolution layer 123 (Conv4) is composed of 18 layers.

The fifth convolution layer 124 (Conv5) analyzes the image pattern via (1×1, 512), (3×3, 512), and (1×1, 2048) filters, and repeats this analysis three times. That is, the fifth convolution layer 124 (Conv5) is composed of 9 layers.

Accordingly, the second neural network 120 is composed of a total of 48 layers.

Similarly, the first neural network 110 is composed of two layers: the first convolution layer 112 (Conv1) and the pooling layer 113.

FIG. 4 is a flowchart showing a process of applying a discriminating model to determine one or more of the cell type, the culturing condition, and the culturing time in the cell discriminating method using artificial intelligence deep learning according to one embodiment of the present disclosure.

Referring to FIG. 4, a discriminating step S200 may include extracting the first feature in the cell image 10 in S210, extracting the second feature in the cell image 10 in S220, and applying the discriminating model 100 based on the first and second features extracted from the cell image 10 to determine one or more of the cell type, the culturing condition, and the culturing time corresponding to the cell image. In this regard, the first feature of the cell image 10 may be large features, for example, a cell shape feature, and the second feature of the cell image 10 may be small features, for example, a cell edge feature.

In the cell discriminating method using artificial intelligence deep learning according to an embodiment of the present disclosure, the various cell types may include animal cells and human cells including any one or more of stem cell lines, human skin fibroblast cell lines, epithelial cell lines, and immune cell lines. The various culturing conditions may be different on each cell line.

The stem cell lines may include any one or more of mouse embryonic stem cell (mES), mouse induced pluripotent stem cell (miPSCs), human embryonic stem cell, human induced pluripotent stem cell, human neural stem cells, human hair follicle stem cells, human mesenchymal stem cells, and human fibroblast cells. The epithelial cell line may include human skin keratinocytes (HaCaT). The immune cell line may include T cells. The human neural stem cells may include human somatic cell-derived cell converted neural stem cells or human brain-derived neural stem cells.

An overview of the culturing conditions in the present disclosure is as follows.

The culturing condition for the mouse embryonic stem cell may include at least one of a culturing condition including LIF (leukaemia inhibitory factor) media, a culturing condition including ITS (insulin-transferrin-selenium supplement) media, and a culturing condition excluding the LIF media. In this regard, the LIF media may function to maintain embryonic stem cell characteristics, and the ITS media may function to induce differentiation.

The culturing condition for the mouse induced pluripotent stem cell may include at least one of a culturing condition including PD0325901 (MEK (mitogen-activated protein kinase) inhibitor), SB431542 (TGF-B (Transforming Growth Factor-β) inhibitor), thiazovivin, ascorbic acid (AA) and LIF media, a culturing condition excluding PD0325901, SB431542, thiazovivin, ascorbic acid, and LIF media, and a culturing condition including ITS media. In this regard, four small molecule compounds, that is, PD0325901, SB431542, thiazovivin, and ascorbic acid function to maintain the characteristics and chromosome stability of mouse induced pluripotent stem cells.

The culturing condition for the human embryonic stem cell or the human induced pluripotent stem cell may include any one or more of a culturing condition including PD0325901, SB431542, thiazovivin, ascorbic acid, and LIF media, a culturing condition excluding PD0325901, SB431542, thiazovivin, ascorbic acid, and LIF media, and a culturing condition including ITS media. In this regard, four small molecule compounds, that is, PD0325901, SB431542, thiazovivin, and ascorbic acid function to maintain chromosome stability.

The culturing condition for the human somatic cell-derived cell-converted neural stem cells may include any one or more of a culturing condition including DMEM/F12 (Dulbecco's Modified Eagle's Medium), N2 (N2 supplement), B27 (serum supplement), bFGFF (basic fibroblast growth factor), EGF (epidermal growth factor), thiazovivin, valproic acid, Purmorphamine, A8301, SB431542, CHIR99021, Deazaneplanocin A (DZNep), and Azacitidine (5-AZA), a culturing condition including DMEM/F12, N2, B27, bFGF, and EGF, and a culturing condition including DMEM/F12 and ITS media. In this regard, small molecule compounds, that is, thiazovivin, valproic acid, purmorphamine, A8301, SB431542, CHIR99021, DZNep, and 5-AZA function to maintain chromosome stability.

The culturing condition for the human brain-derived neural stem cells may include one or more of a culturing condition including a basic medium, an induced neural stem cell growth supplement and antibiotics, a culturing condition including the basic medium and antibiotics, and a culturing condition including the basic medium, antibiotics, and ITS media.

The culturing condition for the human hair follicle stem cells may include any one or more of a culturing condition in which 10% FBS (Fetal bovine serum), Pen/Strep (Penicillin & Streptomycin), L-glutamine, and streptomycin are contained in DMEM media, and a culturing condition in which ITS media is contained in DMEM media.

The culturing condition for the human mesenchymal stem cells may include any one or more of a culturing condition in which 10% FBS (Fetal bovine serum), NEAA (non-Essemtial Amino Acids), and Pen/Strep are contained in DMEM media, and a culturing condition in which the ITS media is contained in DMEM media.

The culturing condition for the human fibroblast may include a culturing condition in which 10% FBS, Pen/Strep, and NEAA are contained in DMEM media.

The culturing condition for the HaCaT cells may include a culturing condition in which 10% FBS, Pen/Strep, L-glutamine, and streptomycin are contained in DMEM media.

The culturing condition for the T cells may include a culturing condition in which Pen/Strep, beta-mercaptoethanol, and L-glutamine are contained in RPMI (Roswell Park Menorial Institute, USA) 1640 media.

In the cell discriminating method using artificial intelligence deep learning according to an embodiment of the present disclosure, the discriminating model 100 may include a data set for image learning. This data set may include a set of 1,000 training images, a set of 1,500 training images and a set of 2,000 training images, a set of 800 validation images, and a set of 100 test images.

Preferably, as a result of training, the discriminating model 100 was able to adopt a set of 2,000 training images.

### Training Results

Under various culturing conditions, various cell types were cultured, and cell images were acquired in the minimum time unit, that is, 1 hour, 3 hours, 6 hours, 12 hours, and 24 hours, after the cell culturing and then were applied to the discriminating model (also known as CNN model) according to the present disclosure for comparison of training accuracy.

In the data set of the discriminating model, a set of 1,000 training images, a set of 1,500 training images and a set of 2,000 training images were used, and a set of 800 validation images, and a set of 100 test images were commonly used.

### A Set of 1,000 Training Images

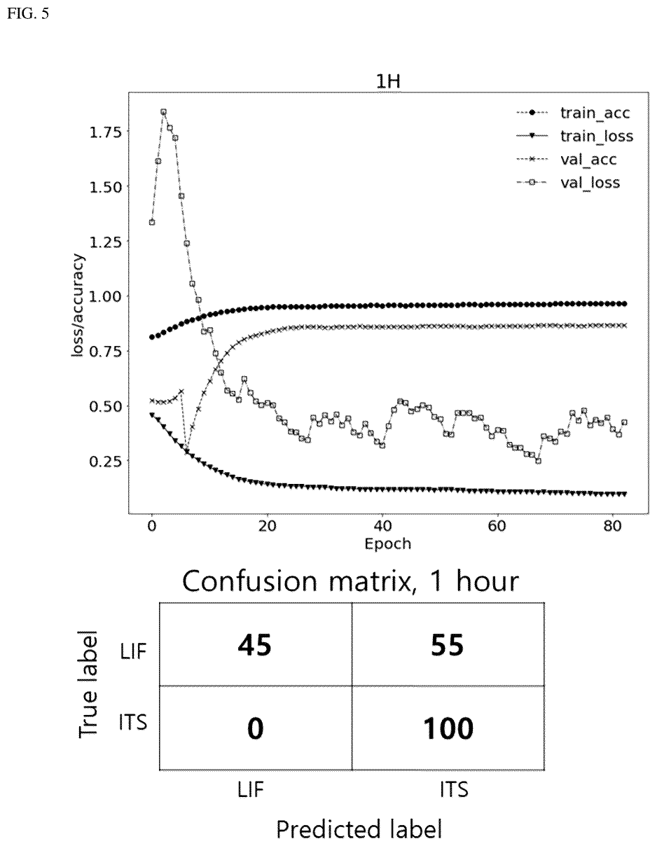

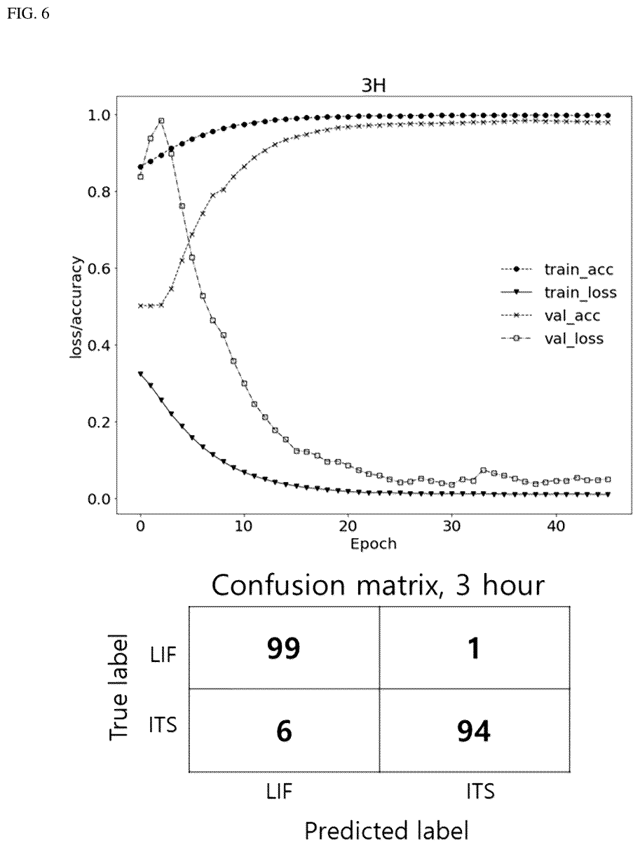

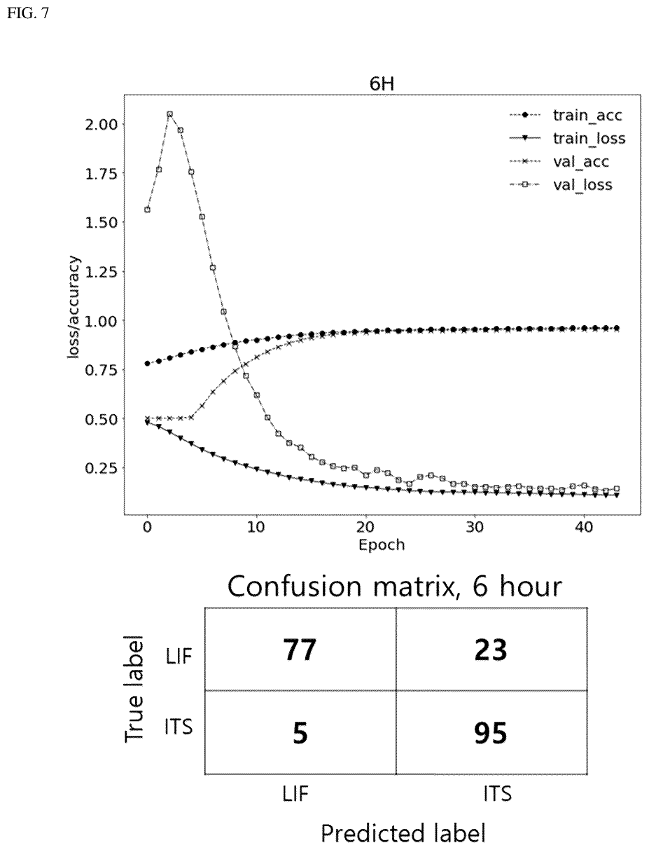

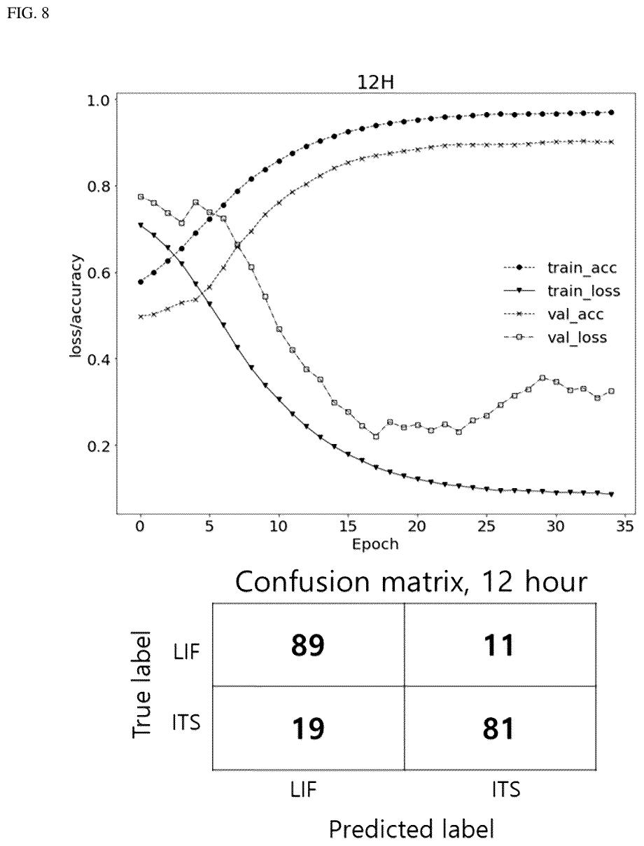

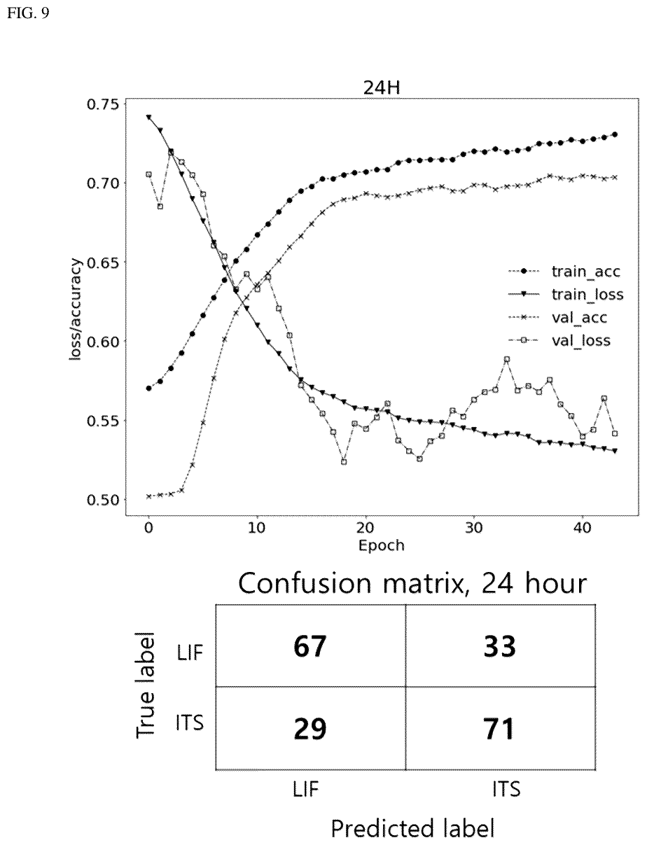

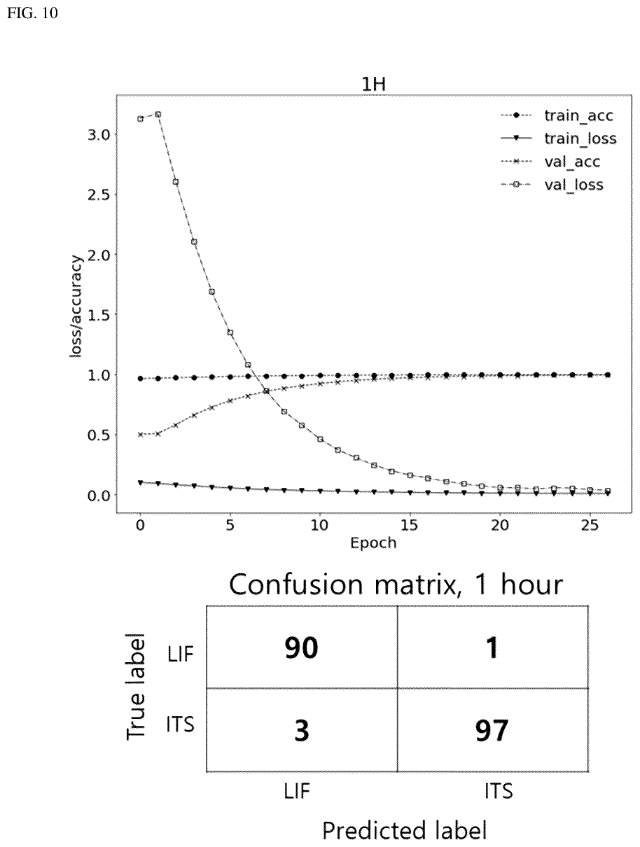

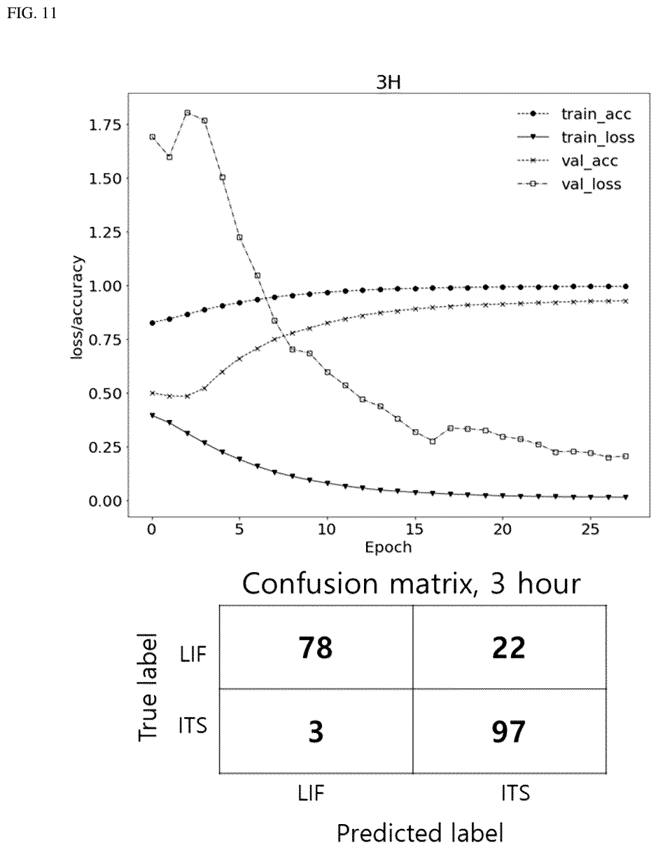

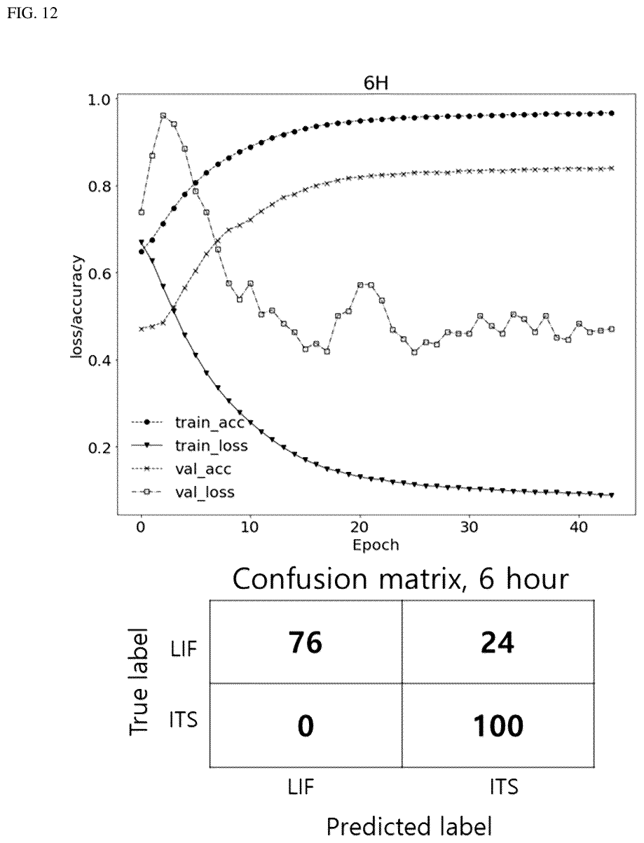

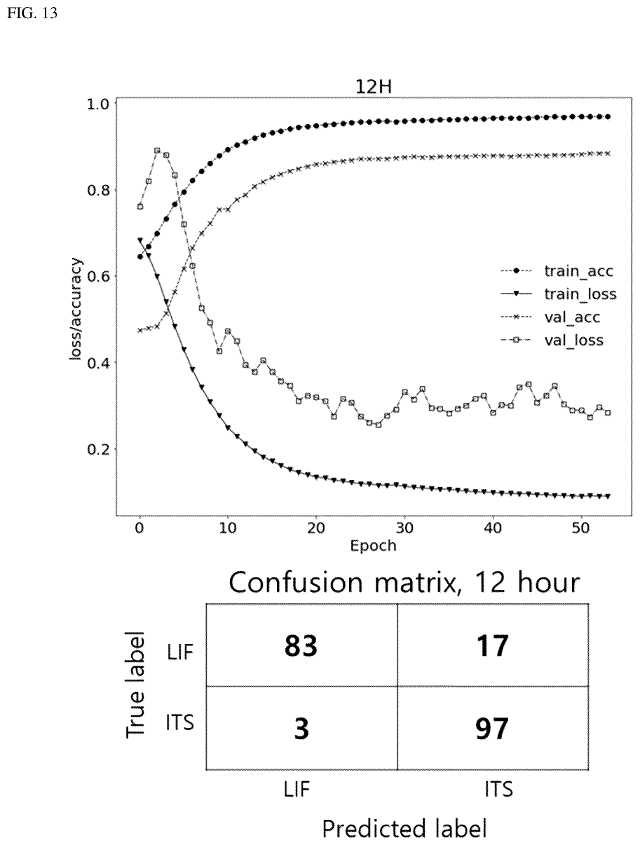

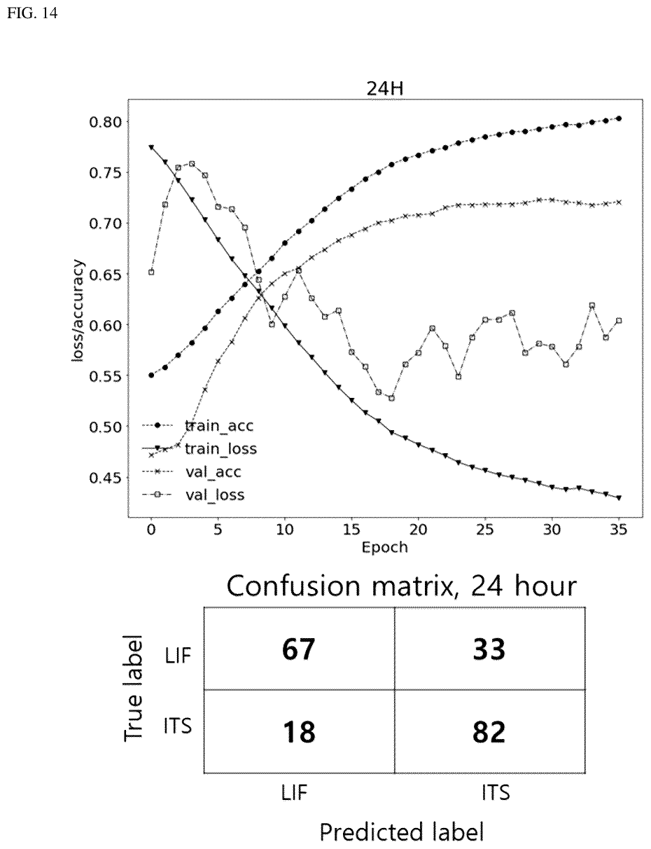

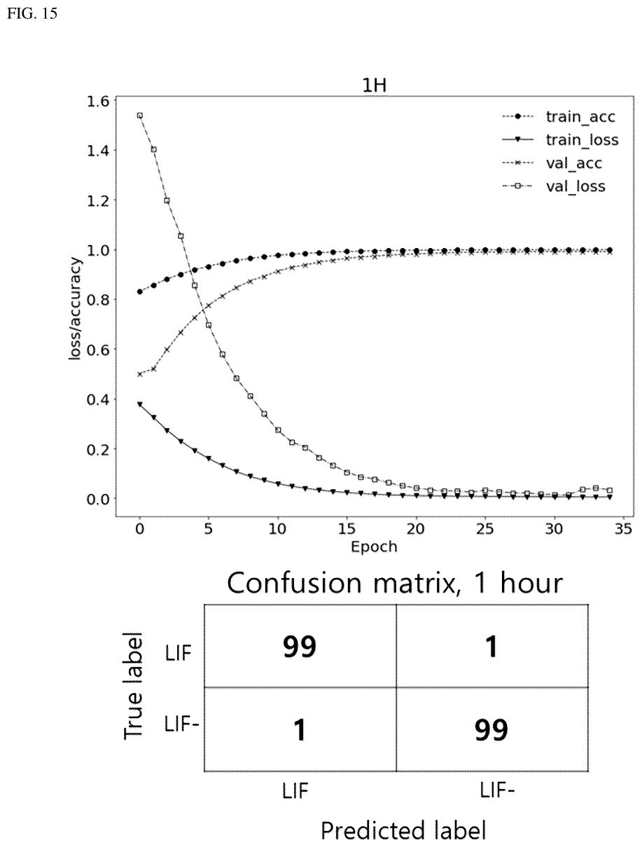

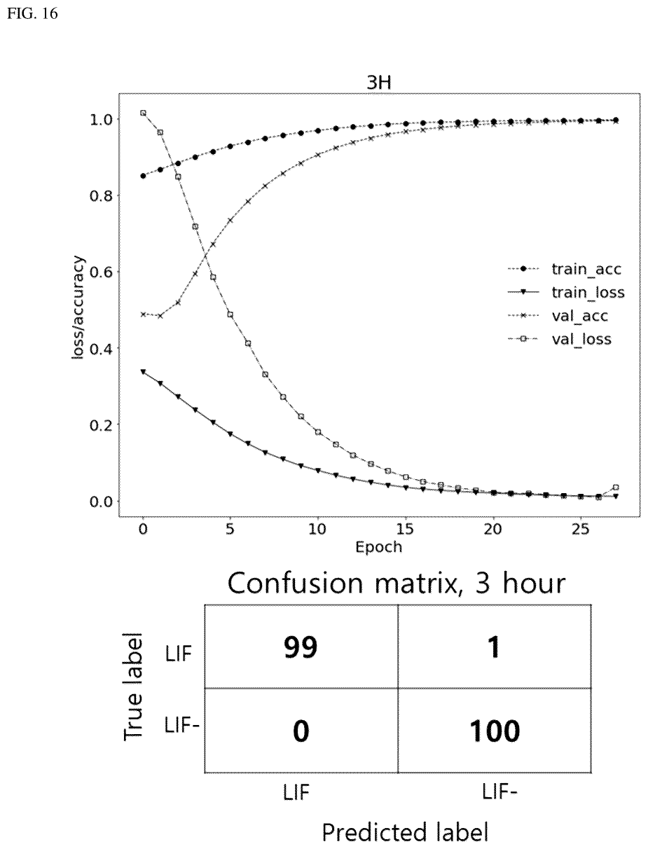

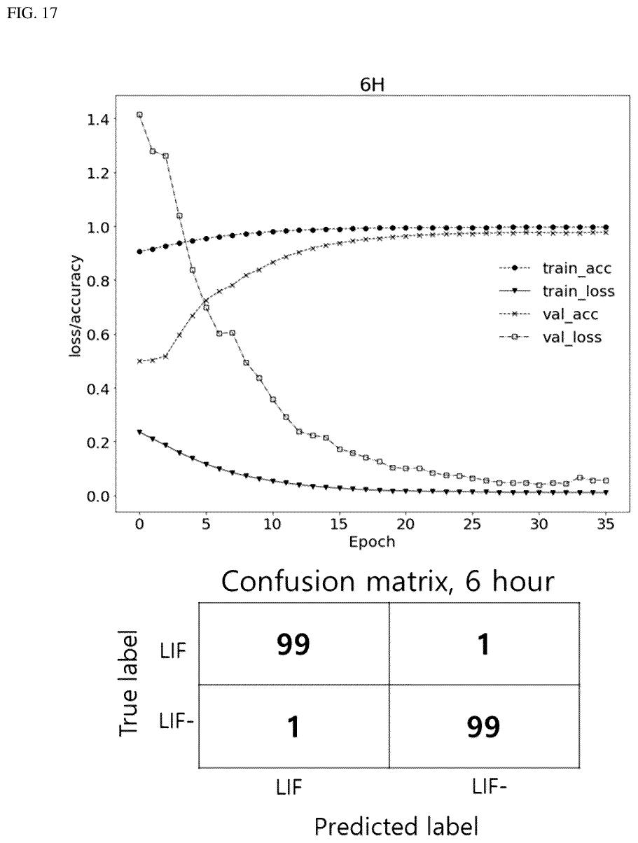

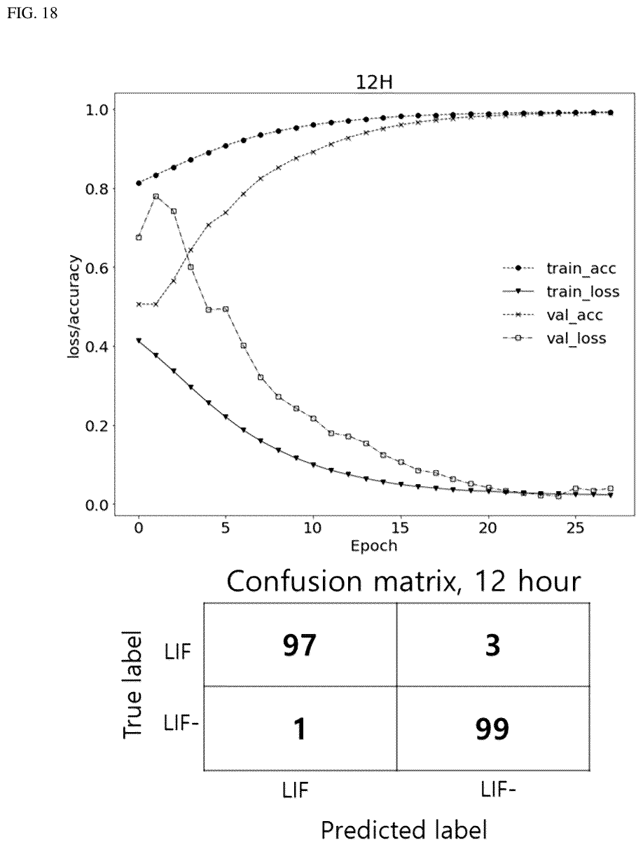

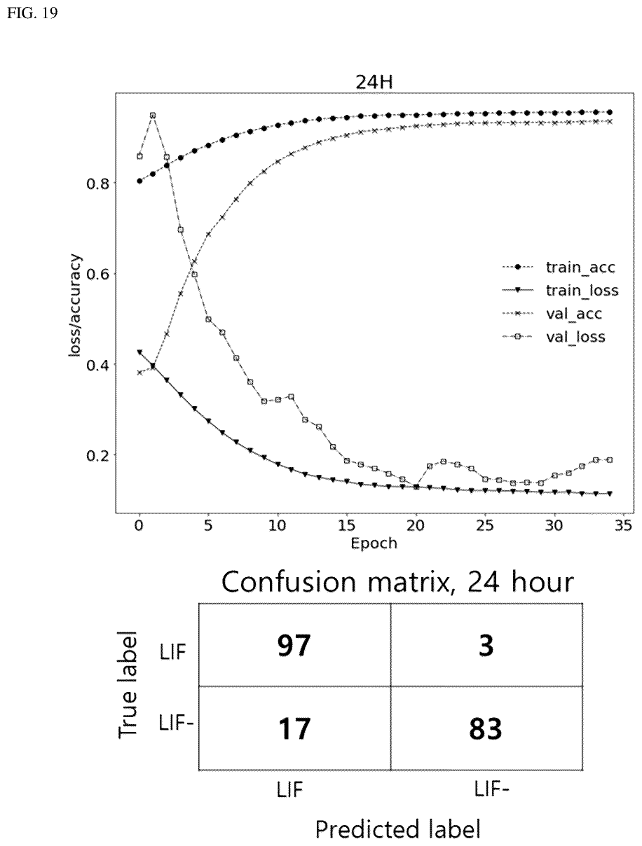

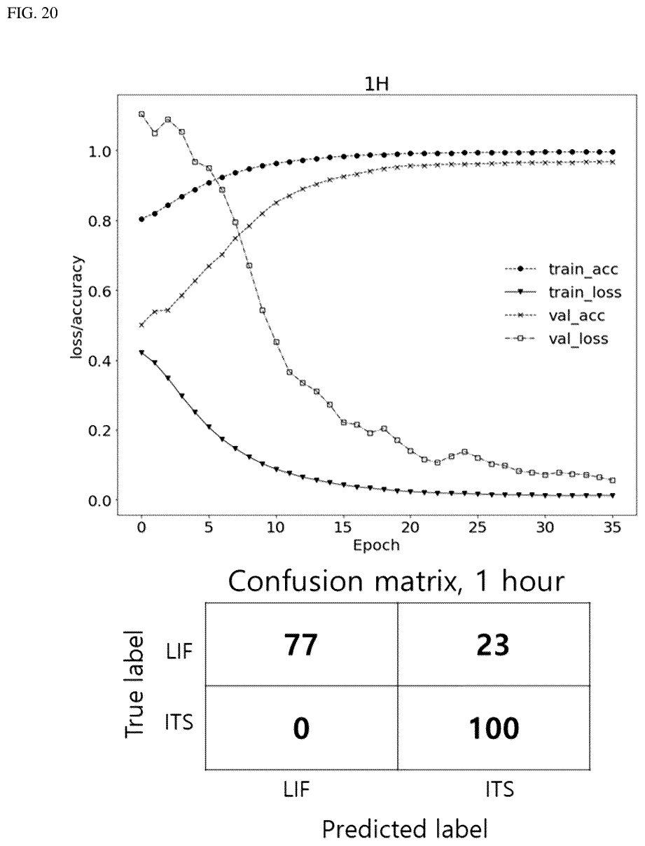

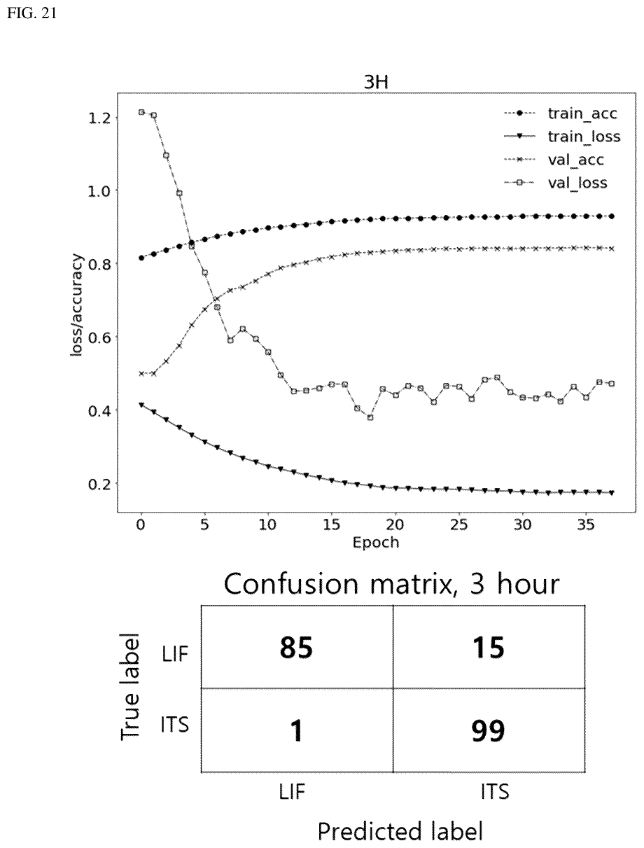

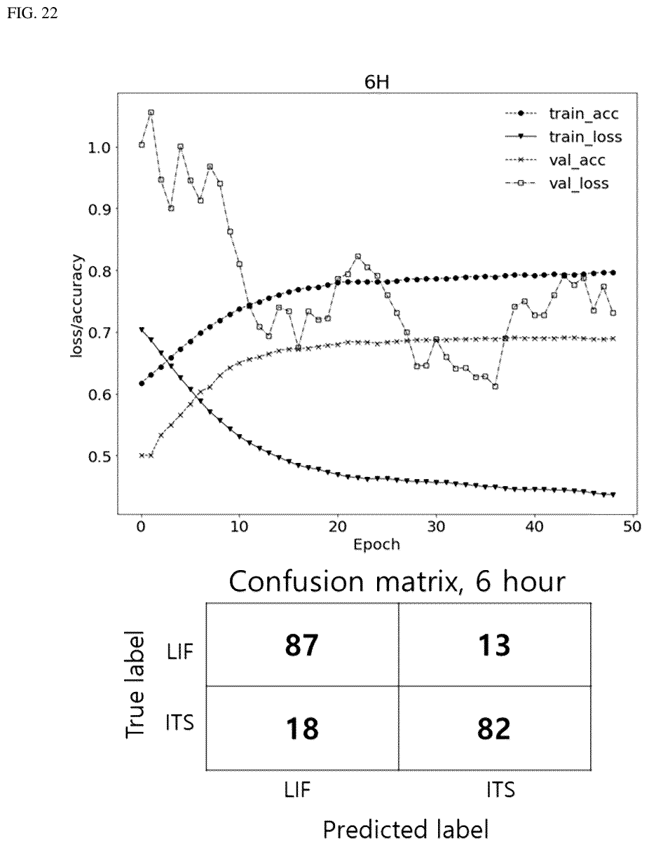

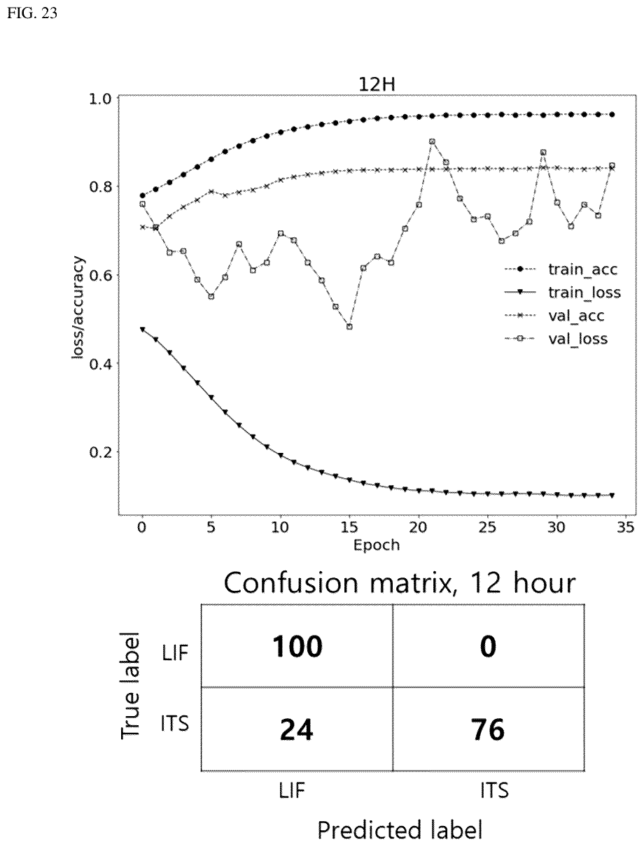

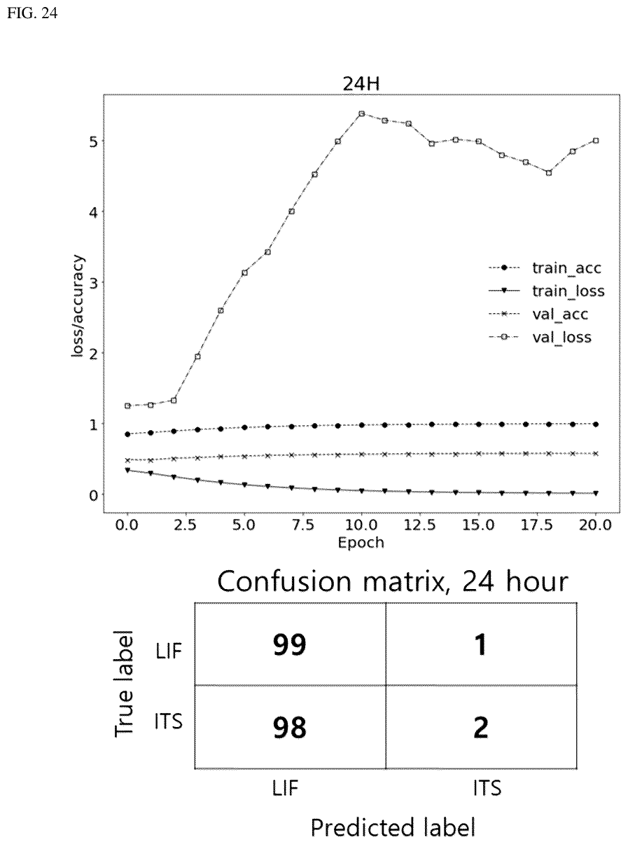

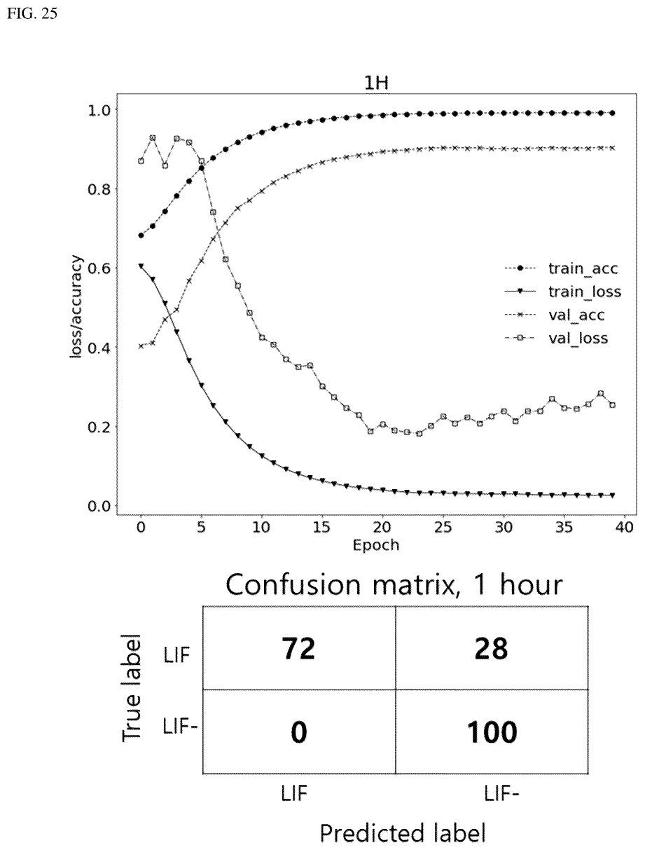

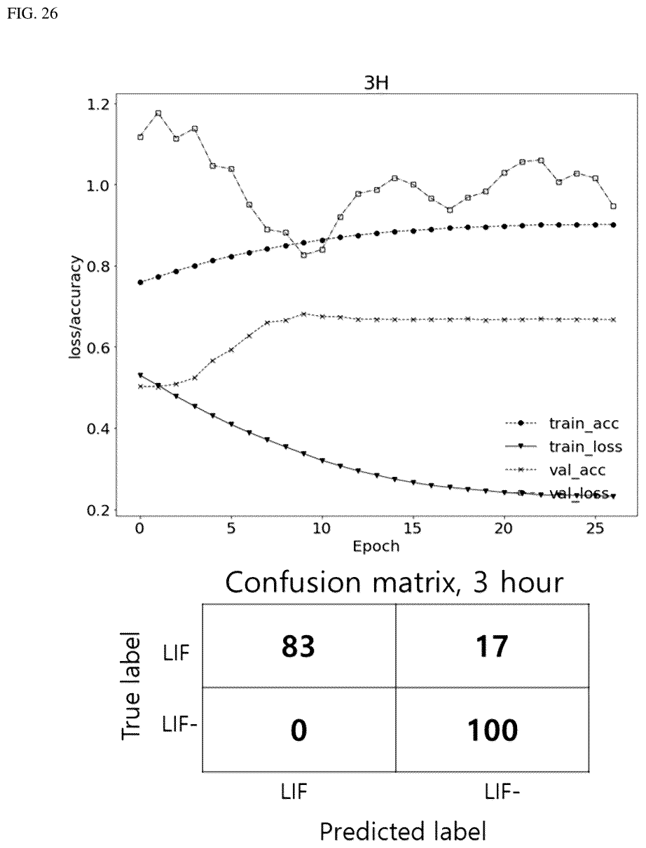

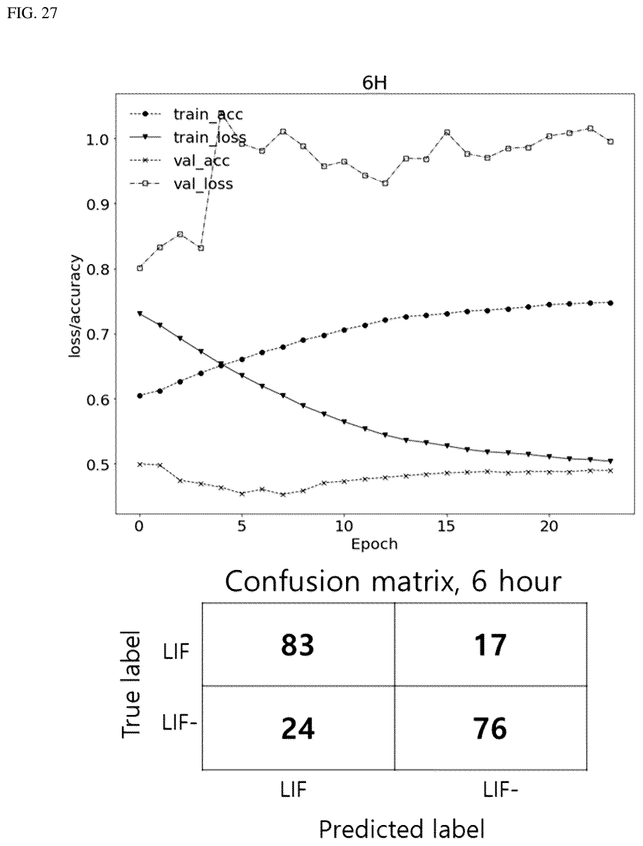

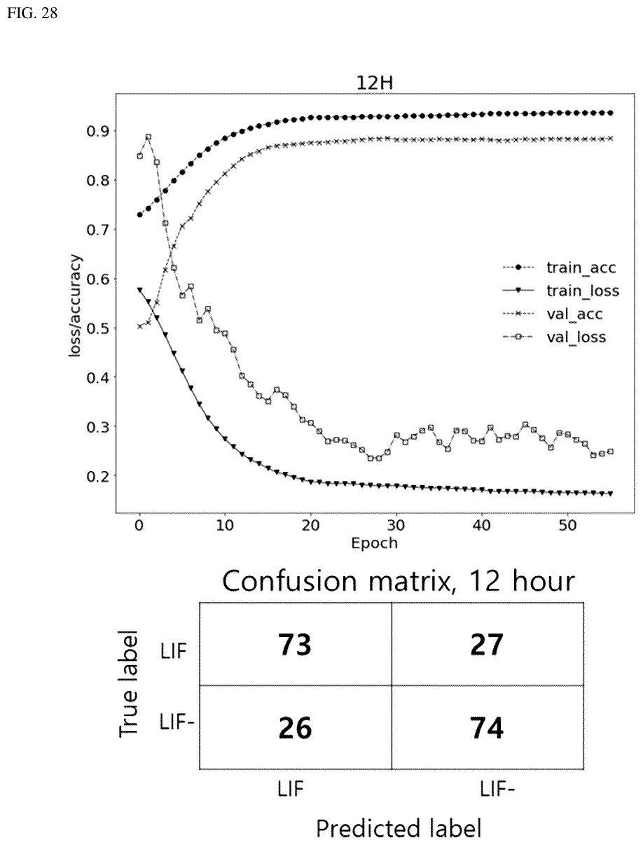

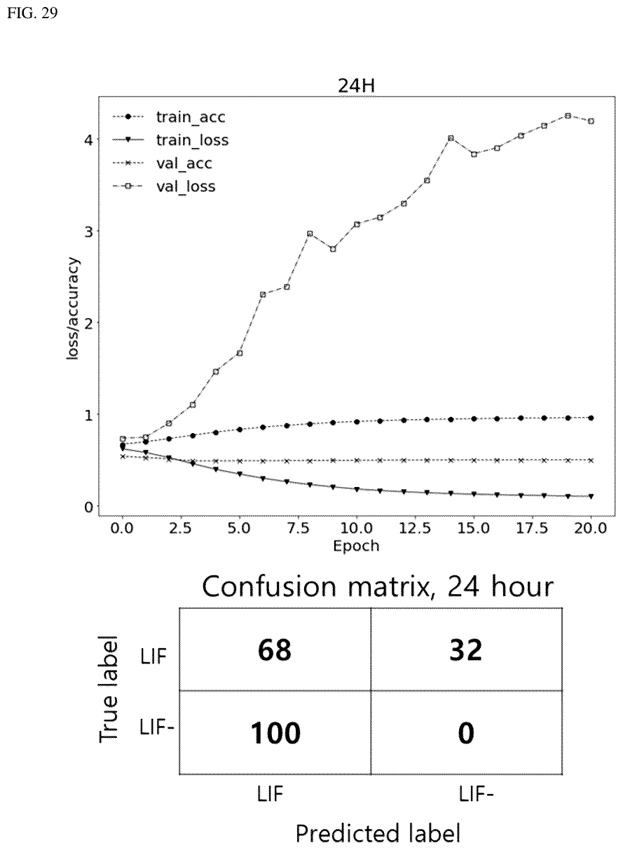

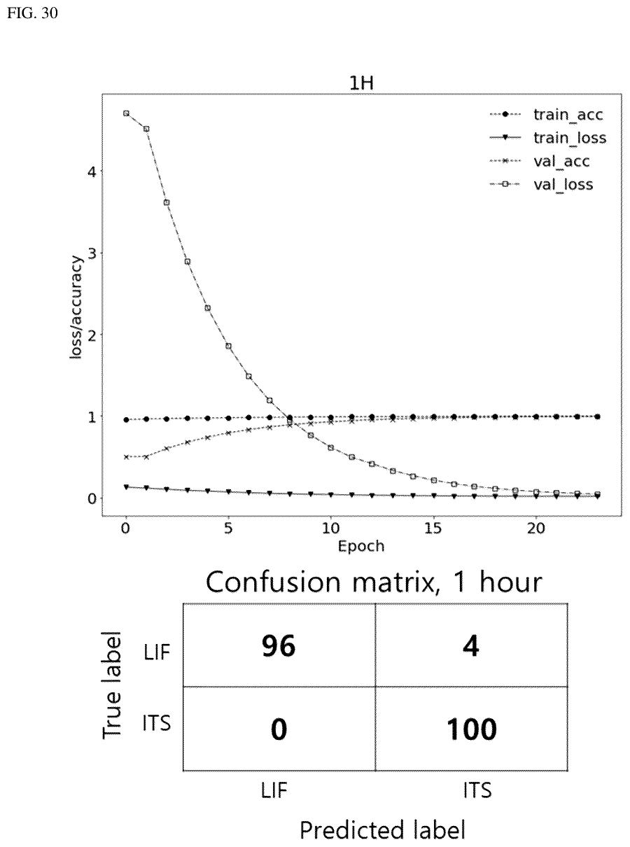

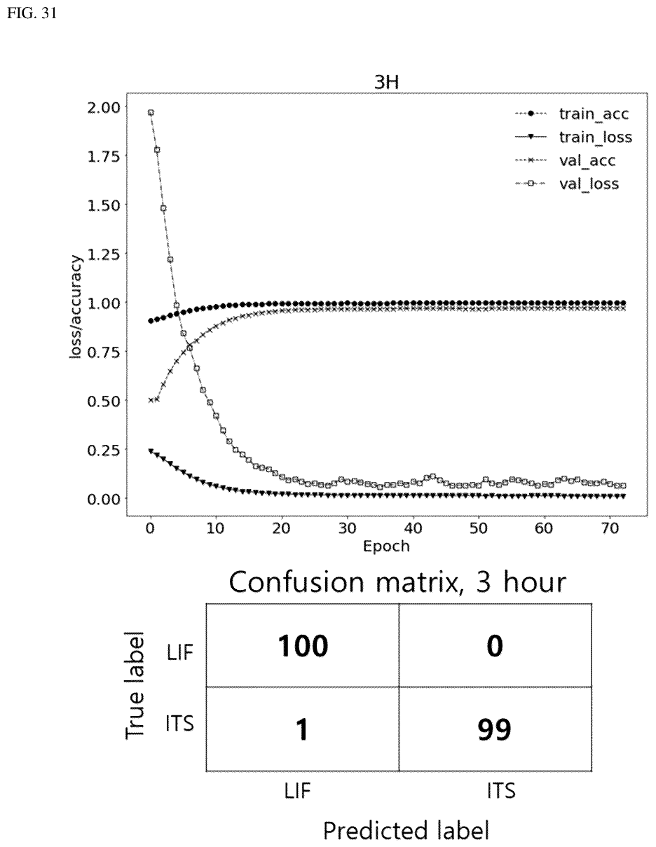

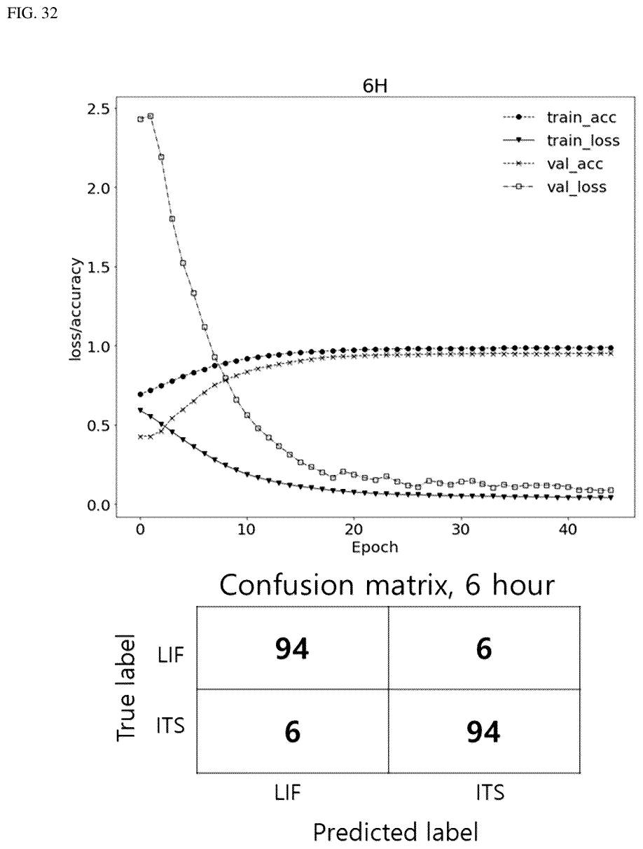

B6 cells (called B6 embryonic stem cells) among mouse embryonic stem cells were cultured using LIF and ITS media. The training results are summarized in FIGS. 5 to 9.

For reference, in the figure, an upper graph shows accuracy and loss (also called a loss value) of each of the training and the validation. The accuracy and loss of the training are expressed as train_acc and train_loss, respectively, and the accuracy and loss of the validation are expressed as val_acc and val_loss, respectively.

Furthermore, a lower confusion matrix is a table showing accuracy using 100 images of the test image set.

In FIGS. 5 to 9, referring to the upper graph, it may be identified that the accuracy of each of the training and the validation is close to 1 in the time zones of 1, 3, and 6 hours, while the accuracy of each of the training and the validation gradually decreases in the time zones of 12 and 24 hours. Furthermore, it may be identified that the loss of each of the training and the validation is generally unstable in all time zones, and in particular, increases to a value more than 0.5 in the time zone of 24 hours.

In FIGS. 5 to 9, referring to the lower confusion matrix, accuracy exceeding 80% was exhibited in all time zones except for the time zone of 24 hours. However, the learning result of this model did not exhibit high accuracy.

As mentioned above, as a result of training using the set of 1,000 training images, the training accuracy was not high.

### A Set of 1,500 Training Images

B6 cells (called B6 embryonic stem cells) among mouse embryonic stem cells were cultured using LIF and ITS media, and were cultured using media to which LIF was added and media from which LIF was removed (marked LIF−). The training results are summarized in FIGS. 10 to 14 and FIGS. 15 to 19.

Furthermore, the mouse induced pluripotent stem cells (miPSCs−) were cultured using LIF and ITS media, and were cultured using media to which LIF was added and media from which LIF was removed (marked LIF−). The training results are summarized in FIGS. 20 to 24 and FIGS. 25 to 29. For reference, the “-” mark in “miPSCs-” means that the four chemicals were not added to the media in the culturing condition.

As described above, as a result of training using the set of 1,500 training images, the accuracy of cell discrimination according to the media in the B6 embryonic stem cells is evaluated to be high to the some extent, but is not reliable enough to perform accurate cell discrimination in actual cell culturing.

Furthermore, in training relate to the induced pluripotent stem cells, the validation loss overfits and increases. Thus, this may be identified as poor training.

Accordingly, the training was conducted using a set of 2,000 training images.

### A Set of 2,000 Training Images

B6 cells (called B6 embryonic stem cells) among mouse embryonic stem cells were cultured using LIF and ITS media, and were cultured using media to which LIF was added and media from which LIF was removed (marked LIF-). The training results are summarized in FIGS. 30 to 34 and FIGS. 35 to 39.

In FIG. 30 to FIG. 34, referring to an upper graph, it may be identified that the accuracy of each of the training and the validation in all time zones is close to 1, and the loss thereof in all time zones decreases to almost 0. This means that the model learning and validation are successful. A lower confusion matrix also showed higher accuracy results than the initial learning results.

In FIGS. 35 to 39, referring to an upper graph, it may be identified that the accuracy of each of the training and the validation in all time zones is close to 1, and the loss thereof in all time zones is converging to 0. In other words, it may be identified that the training and validation processes are successful. A lower confusion matrix showed an accuracy of over 95% until 12 hours. At 24 hours. The accuracy dropped slightly due to the size of the cell, but exhibited an accuracy of over 90%.

J1 cells (called J1 embryonic stem cells) among mouse embryonic stem cells were cultured using LIF and ITS media, and were cultured using media to which LIF was added and media from which LIF was removed (marked LIF−). The training results are summarized in FIGS. 40 to 44 and FIGS. 45 to 49.

In FIG. 40 to FIG. 44, referring to an upper graph, the accuracy of each of the training and the validation was close to 1. In a lower confusion matrix, the accuracy of distinguishing the cells cultured in the LIF media was 99% or 100%, and the accuracy of distinguishing the cells cultured in the ITS media was over 97%.

In FIGS. 45 to 49, referring to an upper graph, the accuracy of each of the training and the validation was close to 1. In a lower confusion matrix, an accuracy thereof was over 99%.

The mouse induced pluripotent stem cells (miPSCs+Line1) were cultured using LIF and ITS media, and were cultured using media to which LIF was added and media from which LIF was removed (marked LIF−). The training results are summarized in FIGS. 50 to 54 and FIGS. 55 to 59. For reference, the “+” sign in “miPSCs+” means that the four chemicals were added to the culturing media in the culturing condition.

Referring to FIG. 50 to FIG. 54, in an upper graph, the accuracy of each of the training and the validation was close to 1, while the loss of the validation was stable except for the time zones of 1 and 12 hours.

Referring to FIGS. 55 to 59, in an upper graph, the accuracy of each of the training and the validation was generally good, while the loss of the validation was unstable in the time zones of 1 and 24 hours. The biggest reason therefor seems to be the creation of cellular debris and the rapid proliferation rate of cells. The accuracy of the error sorting was over 96% except for the time zone of 24 hours. This is because of limitation in distinguishing the cell shape due to the rapid proliferation rate and the debris of cells in the time zone of 24 hours as described above.

The mouse induced pluripotent stem cells (miPSCs+Line2) were cultured using LIF and ITS media, and were cultured using media to which LIF was added and media from which LIF was removed (marked LIF−). The training results are summarized in FIGS. 60 to 64 and FIGS. 65 to 69.

Referring to FIGS. 60 to 64, in an upper graph, the accuracy of each of the training and the validation was close to 1, while the loss of the validation was close to 0 in all time zones, except that the loss thereof was slightly unstable in the time zone of 1 hour. In a lower the confusion matrix, the accuracy was over 98%. The accuracy of distinguishing the cells cultured in the ITS media was over 93%, and was reliable.

Referring to FIGS. 65 to 69, in an upper graph, the accuracy of each of the training and the validation was closer to 1, except that the loss of the validation was slightly unstable in the time zone of 24 hours. Even in the confusion matrix, the accuracy of distinguishing cells cultured in each media was very high in all time zones.

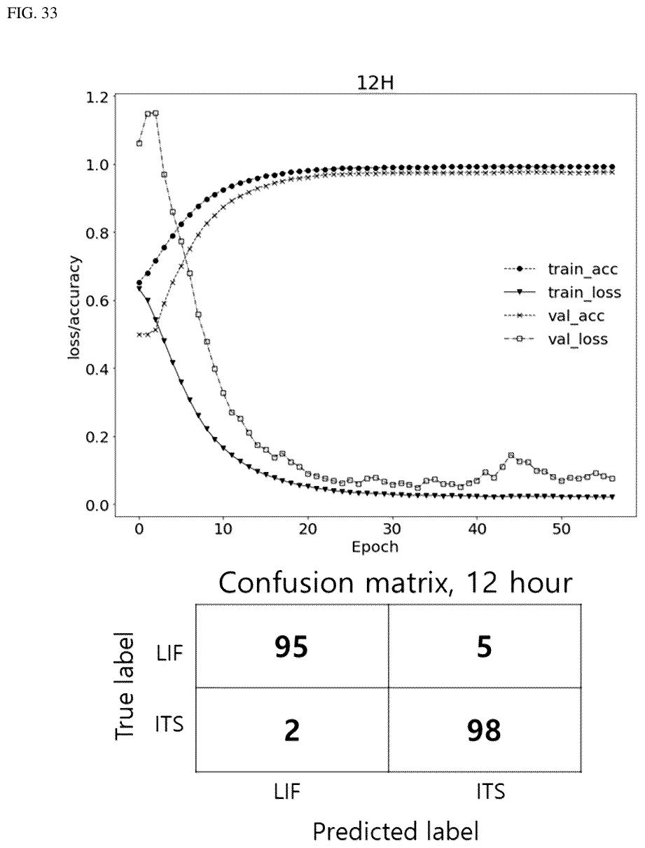

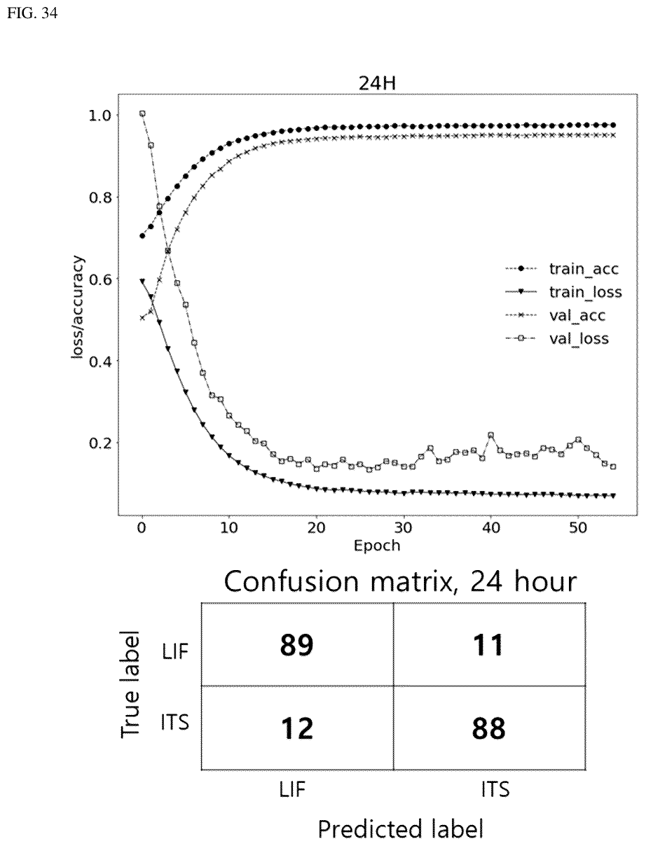

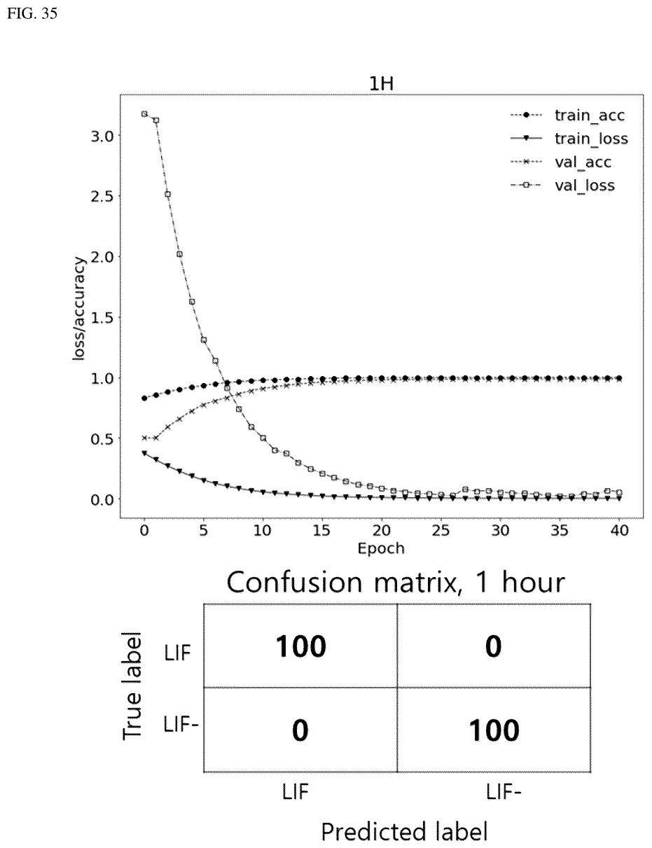

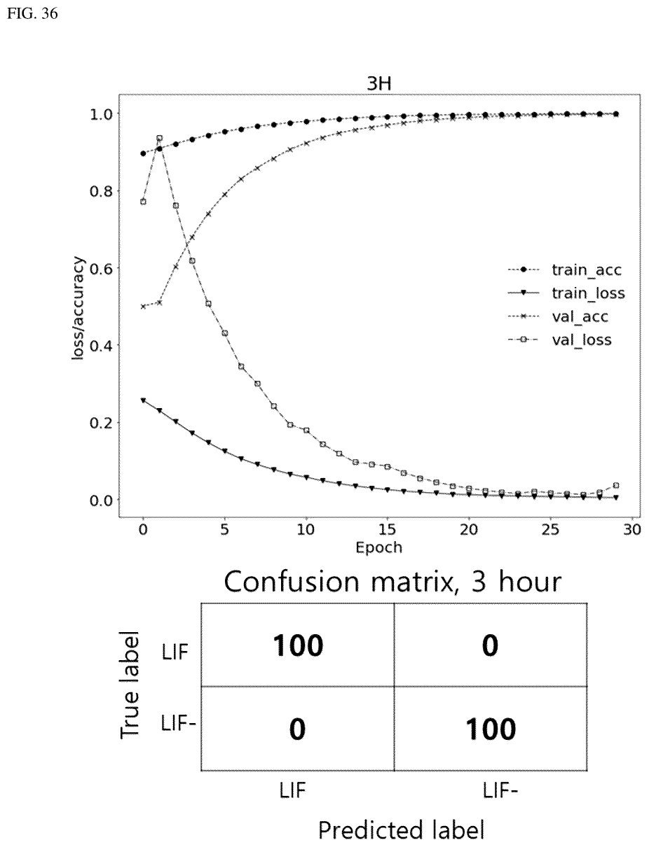

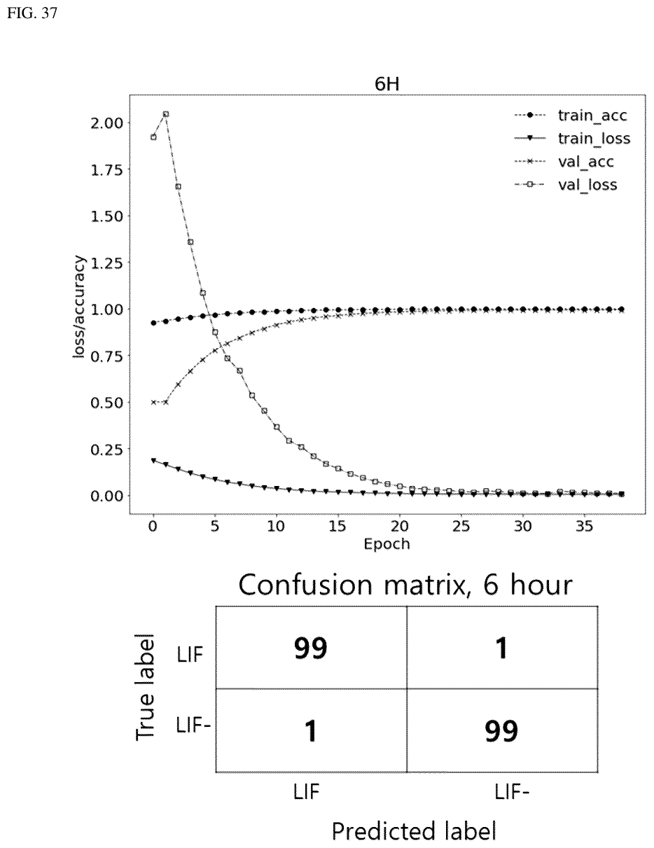

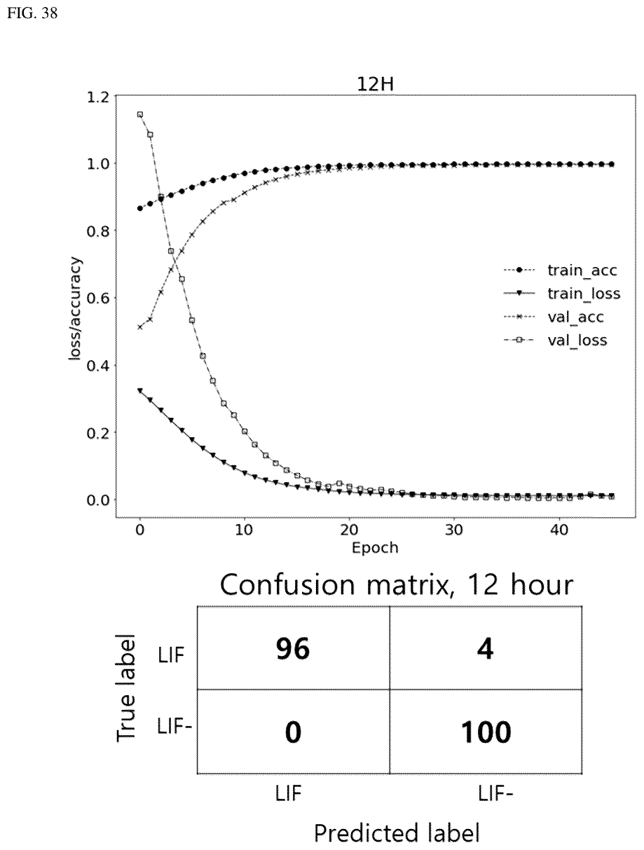

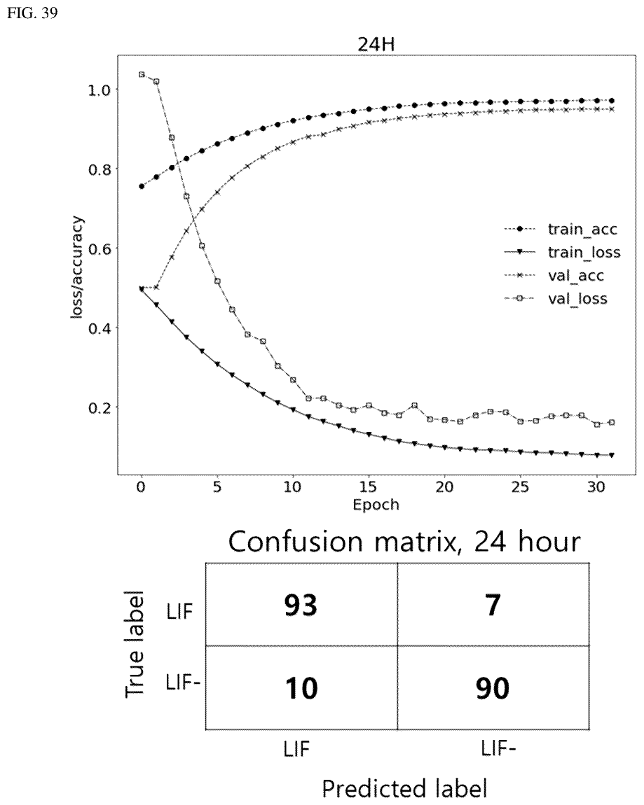

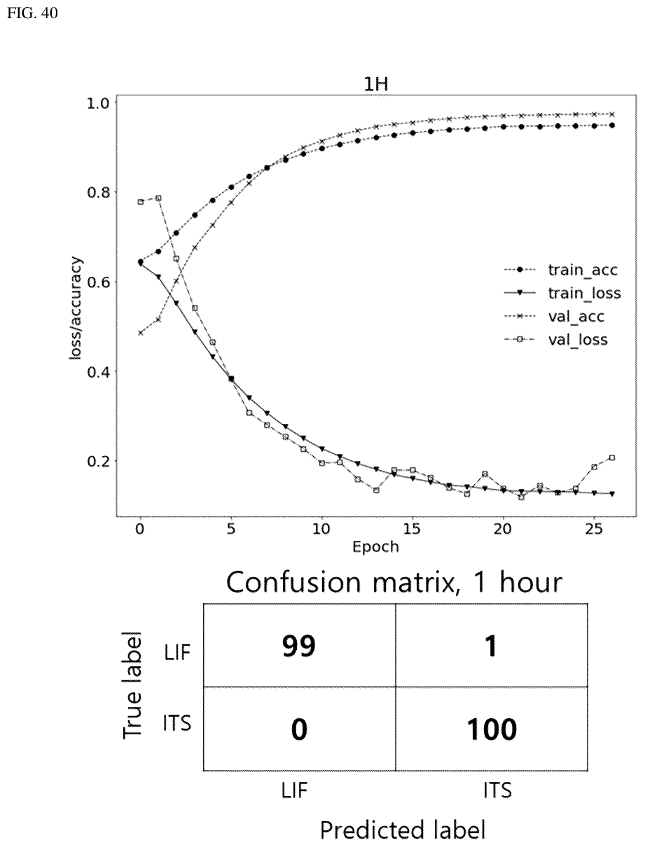

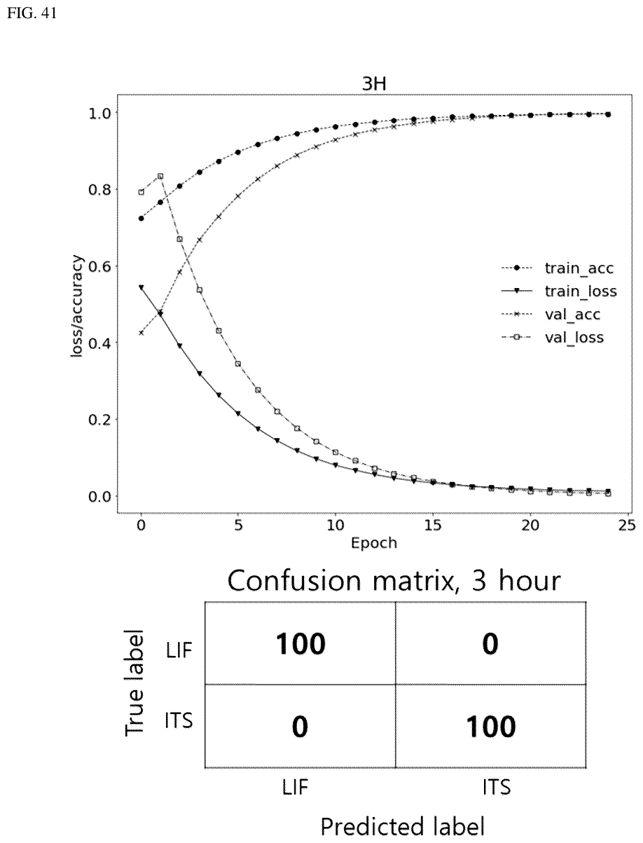

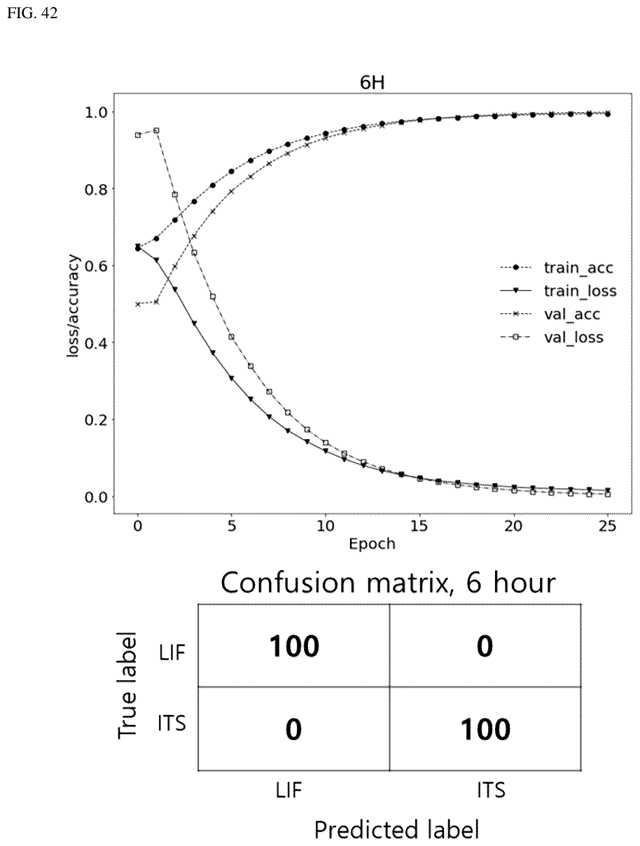

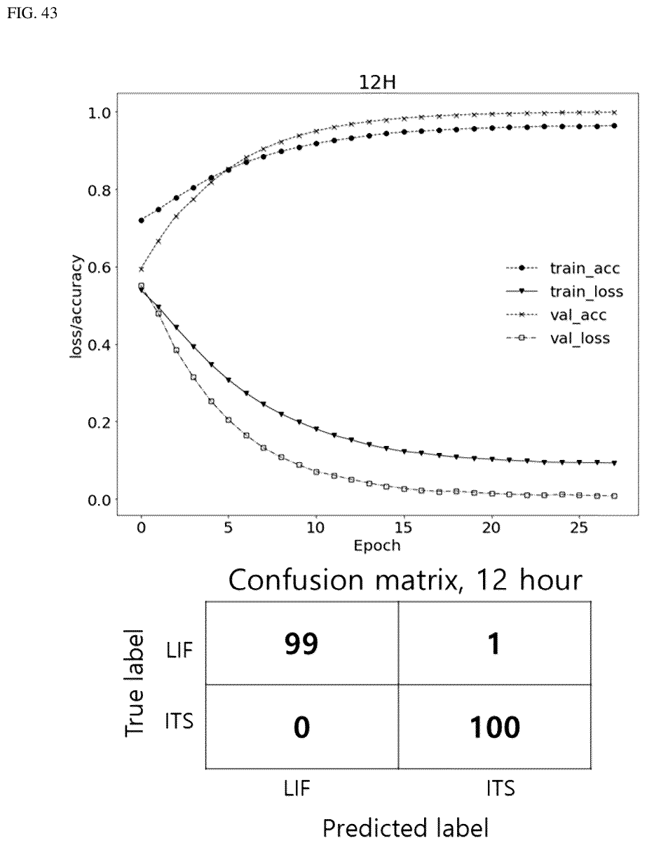

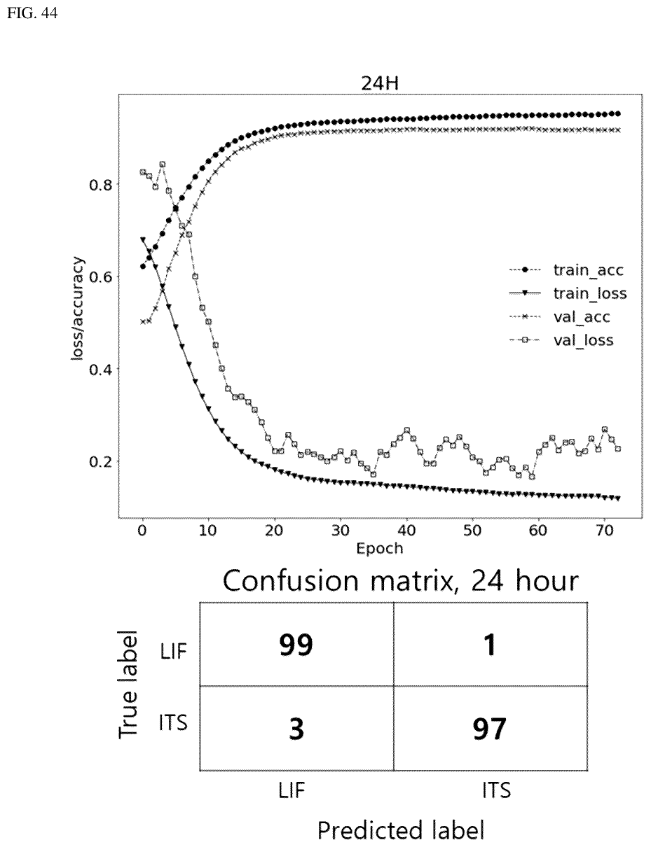

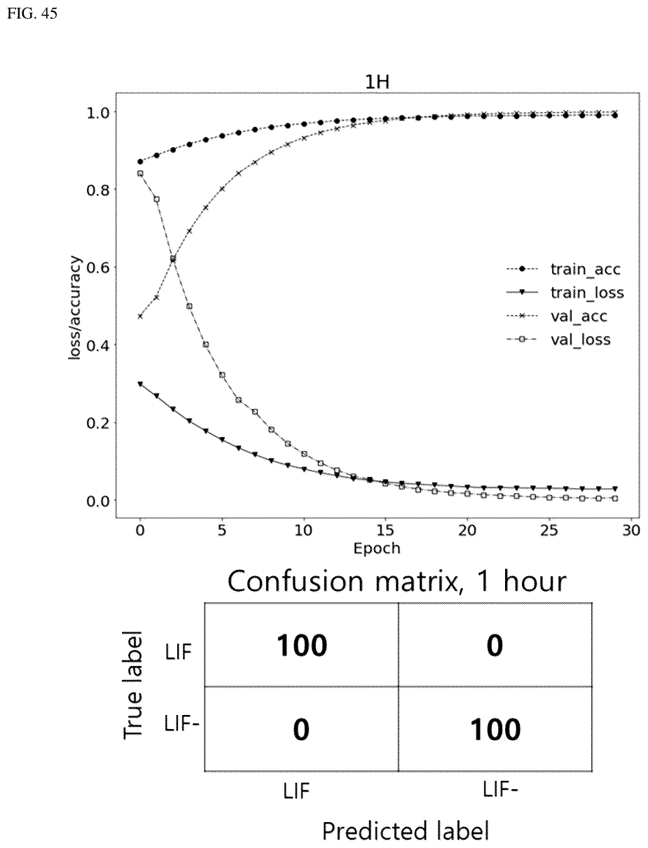

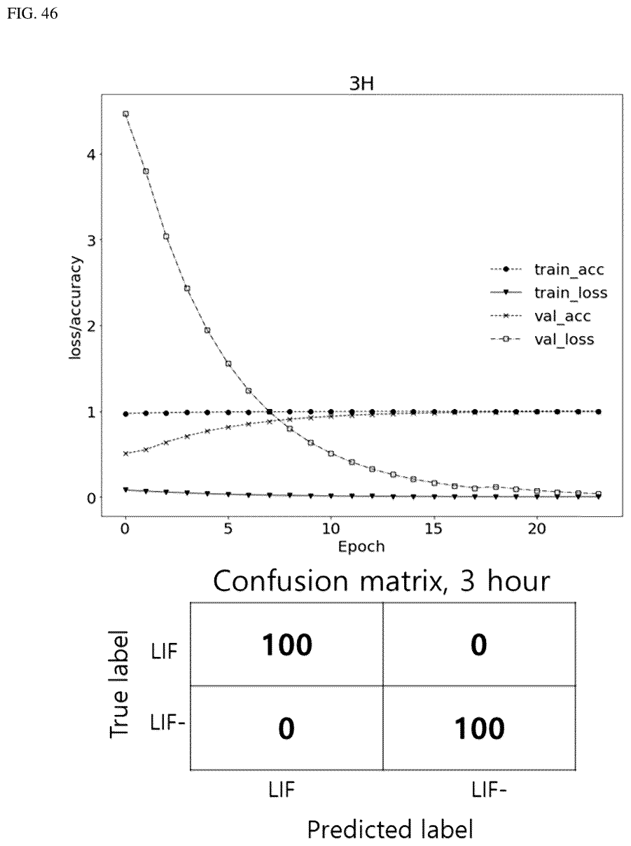

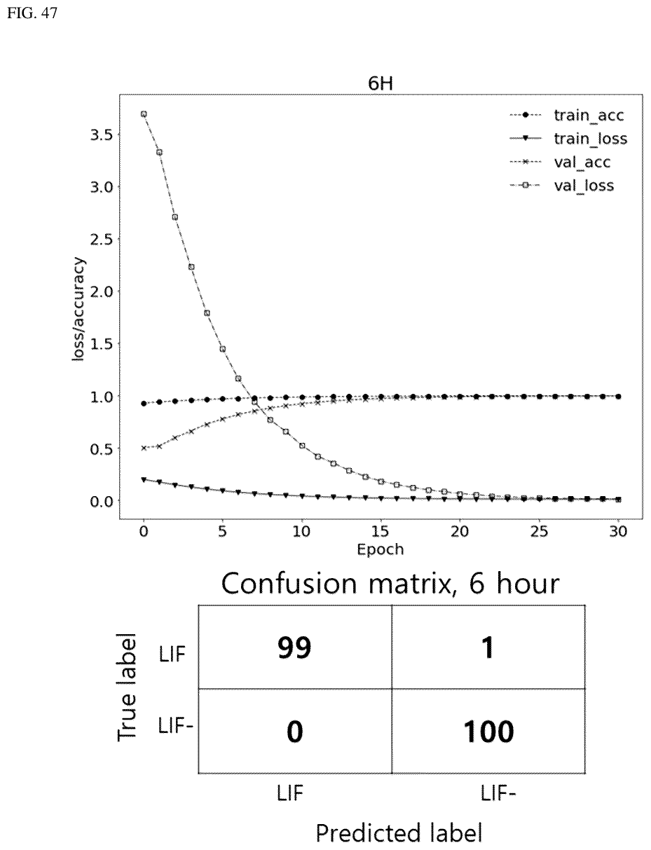

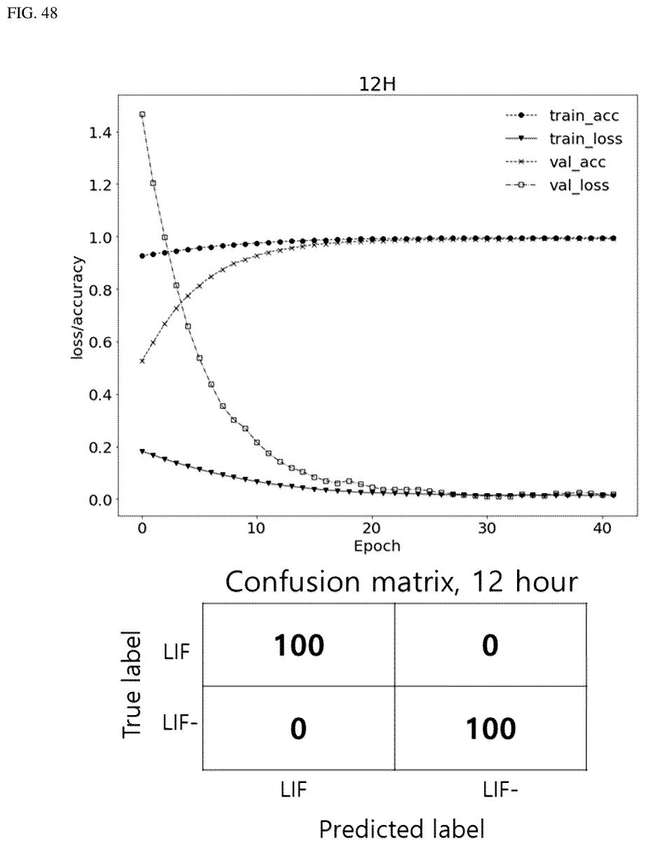

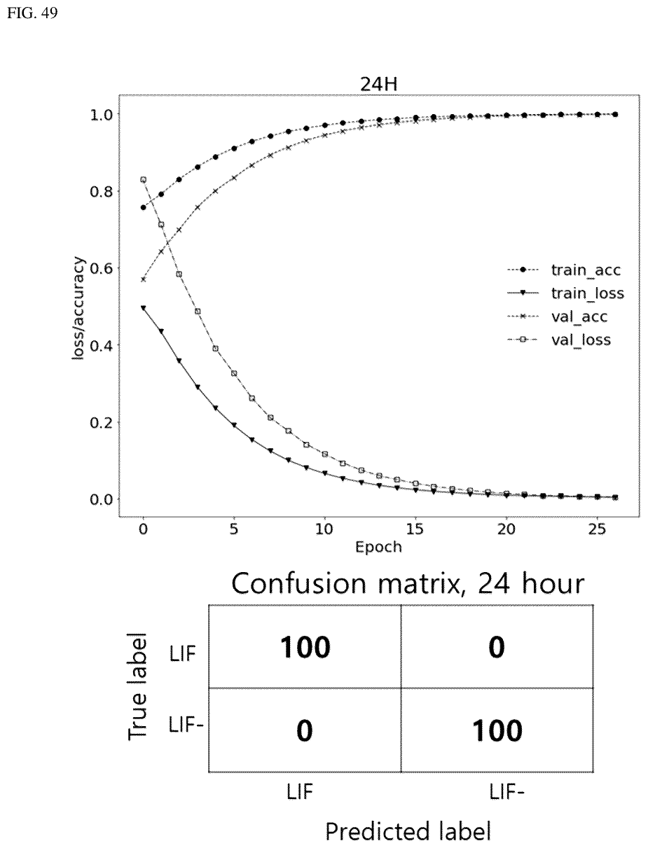

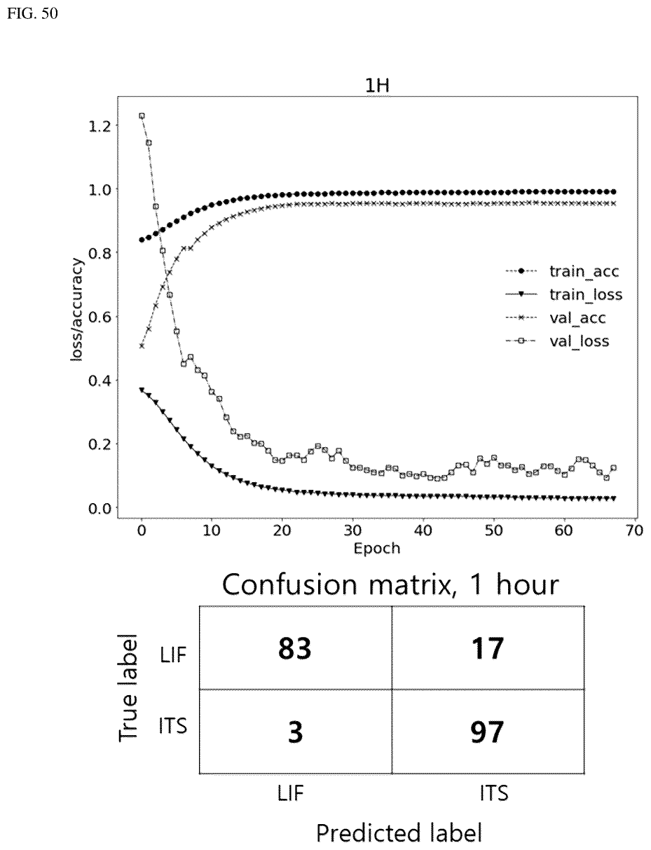

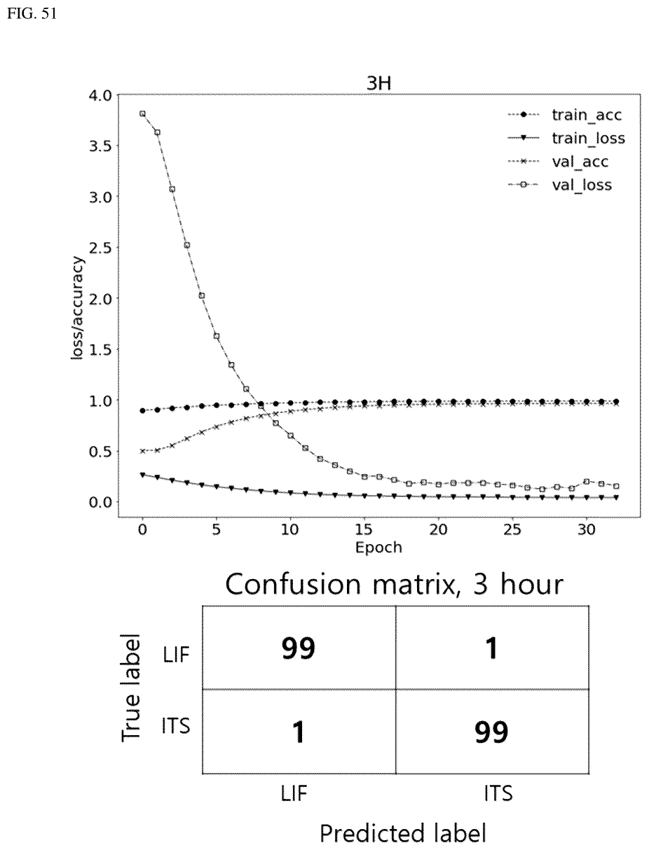

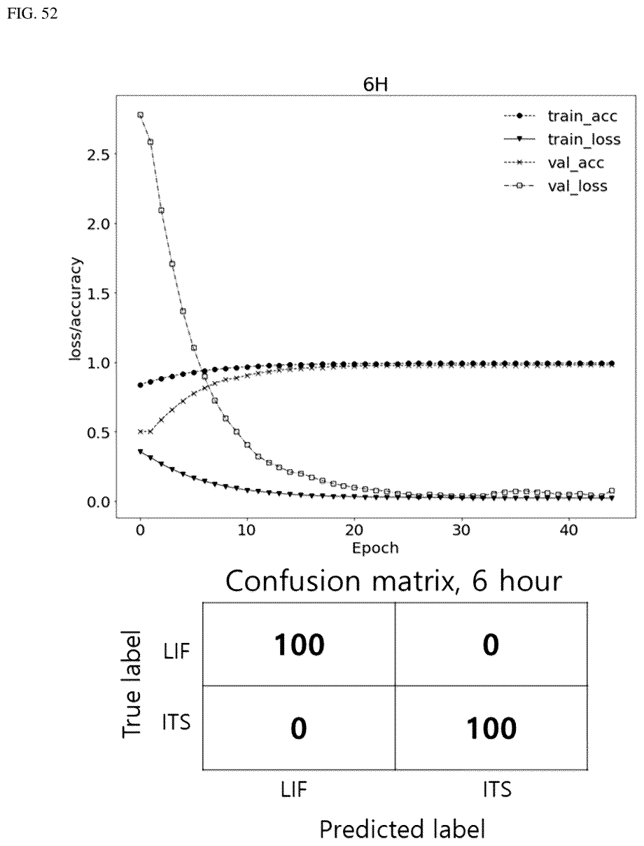

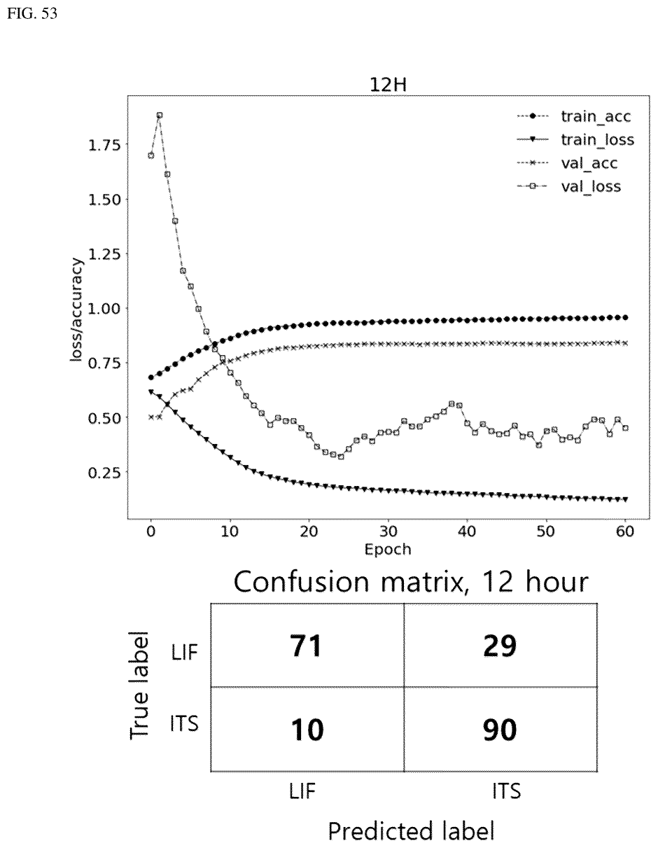

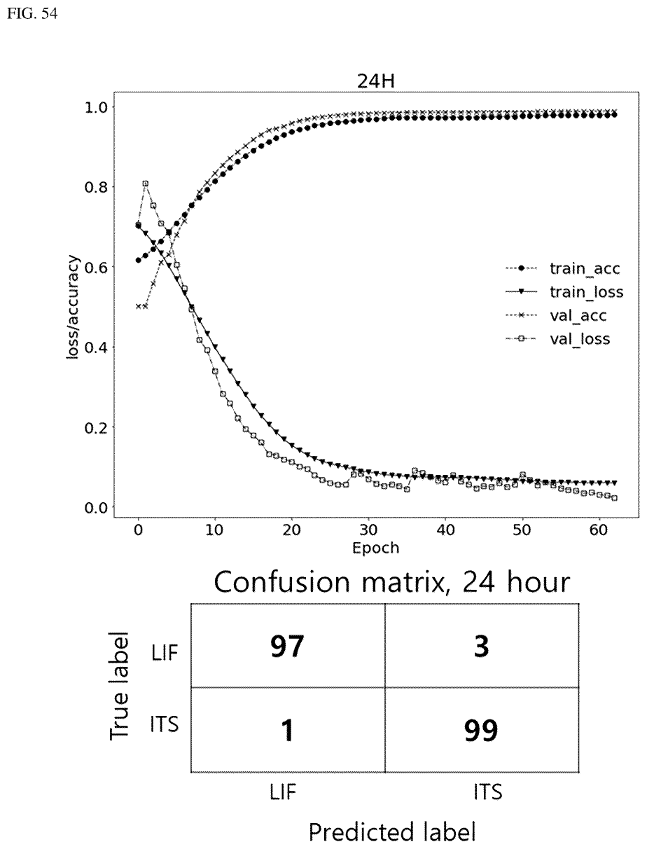

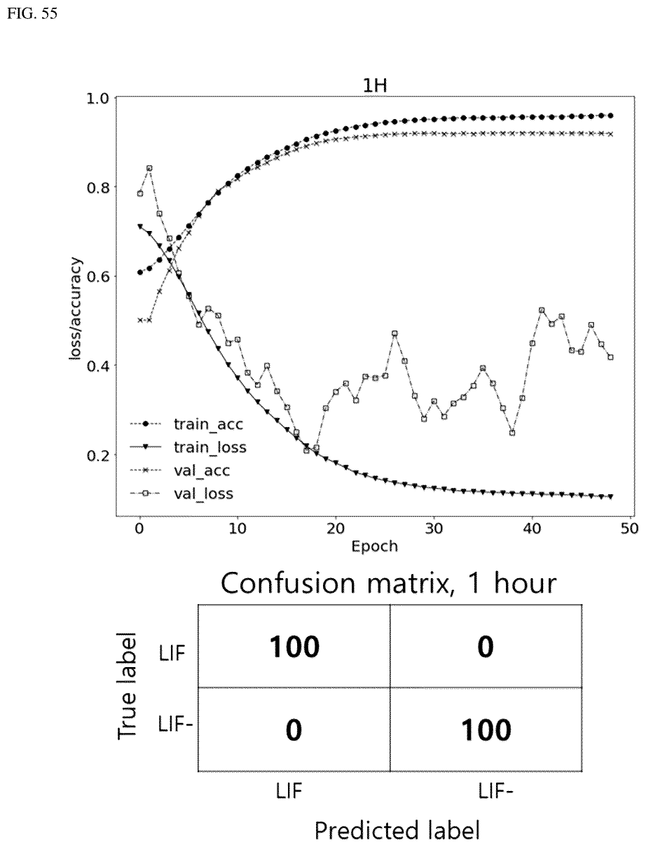

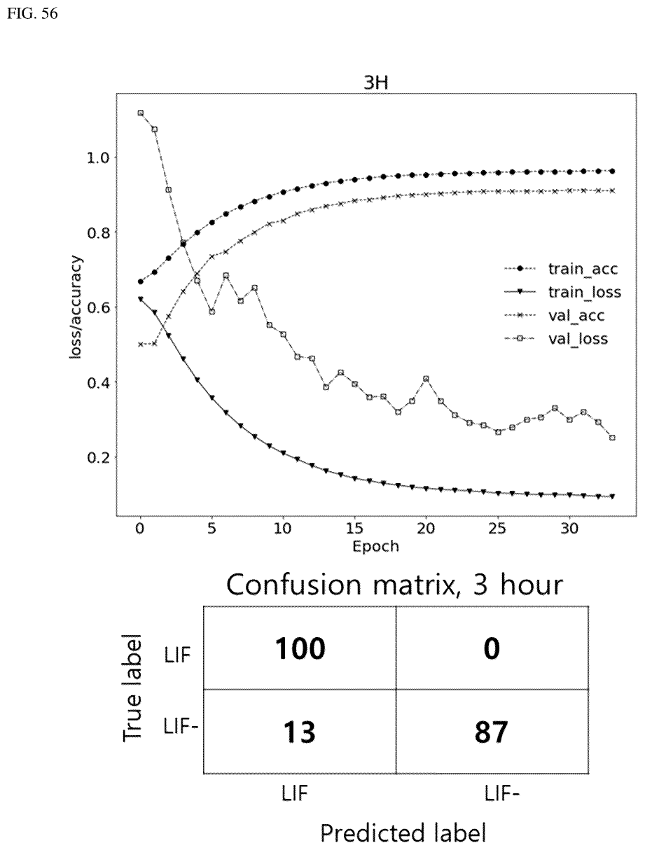

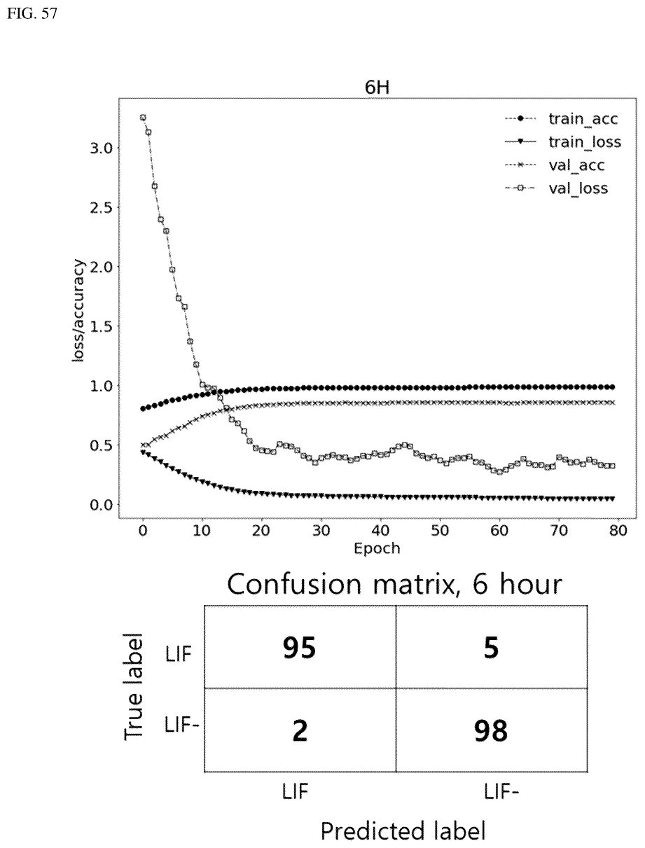

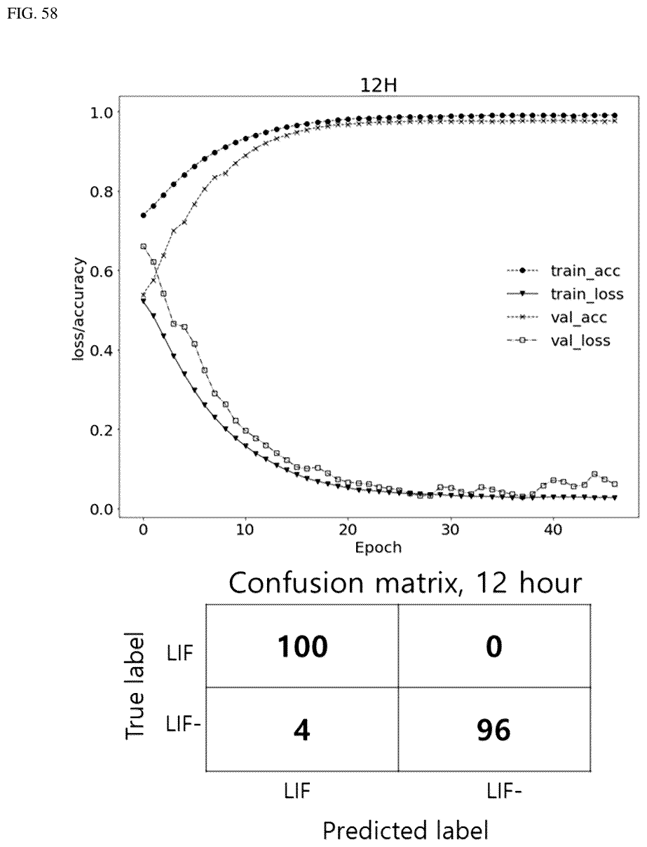

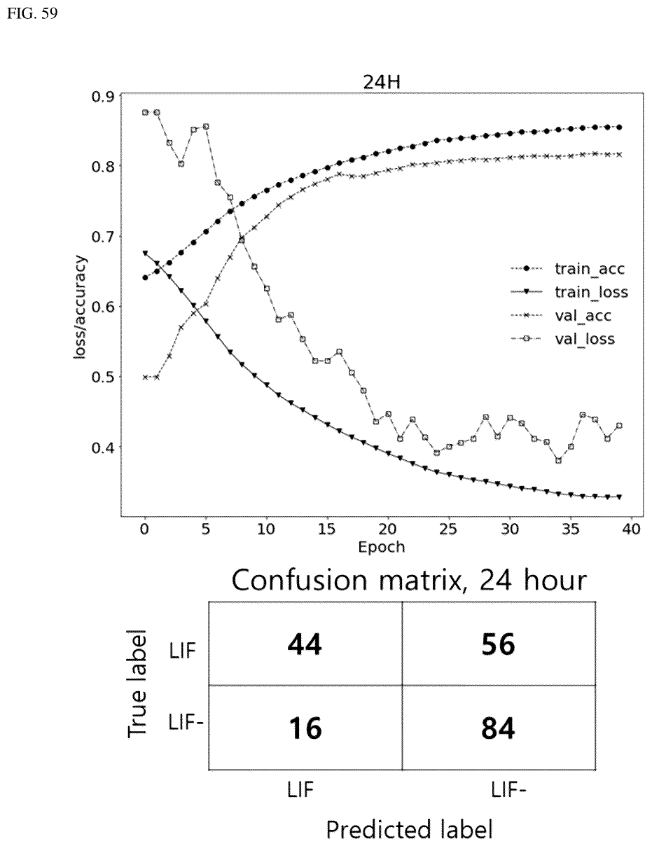

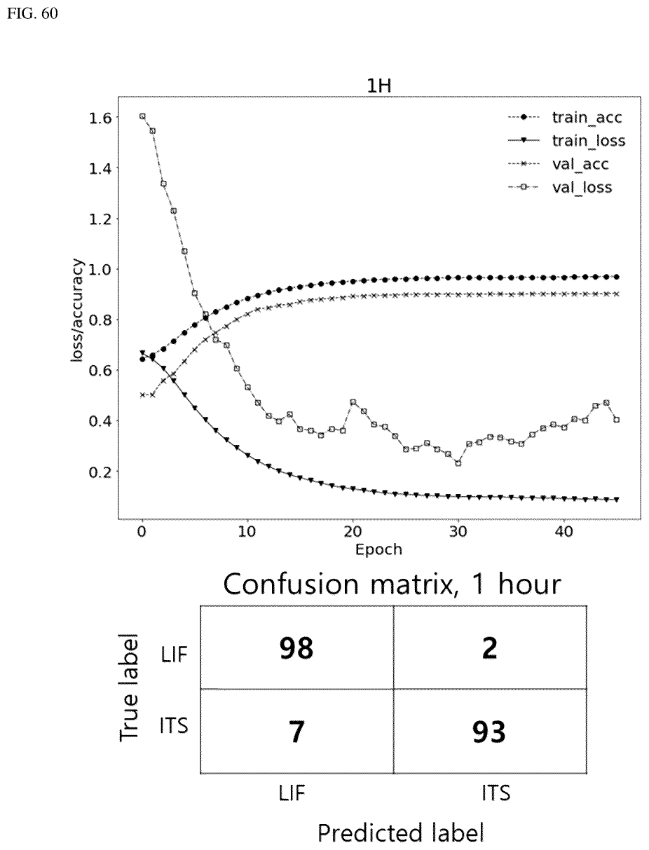

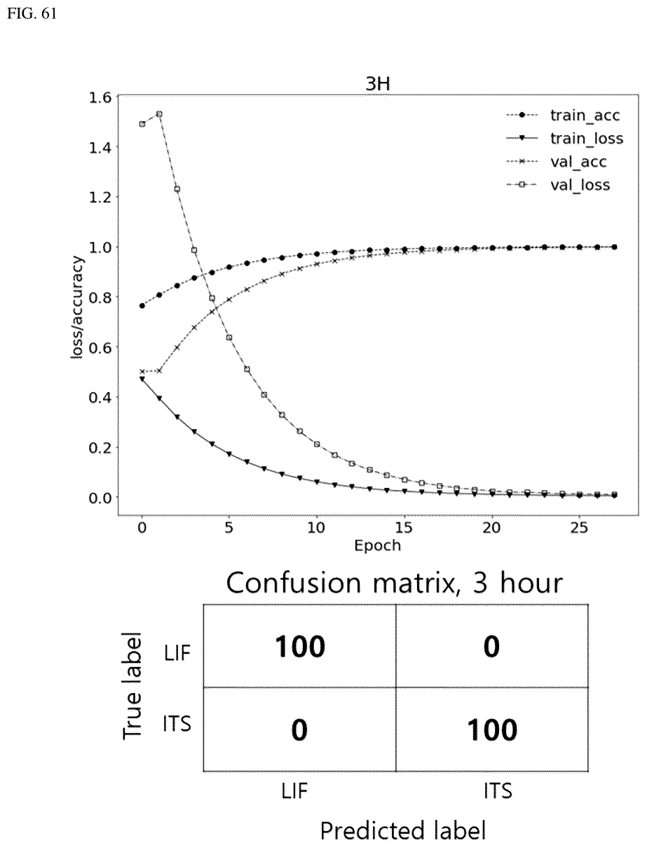

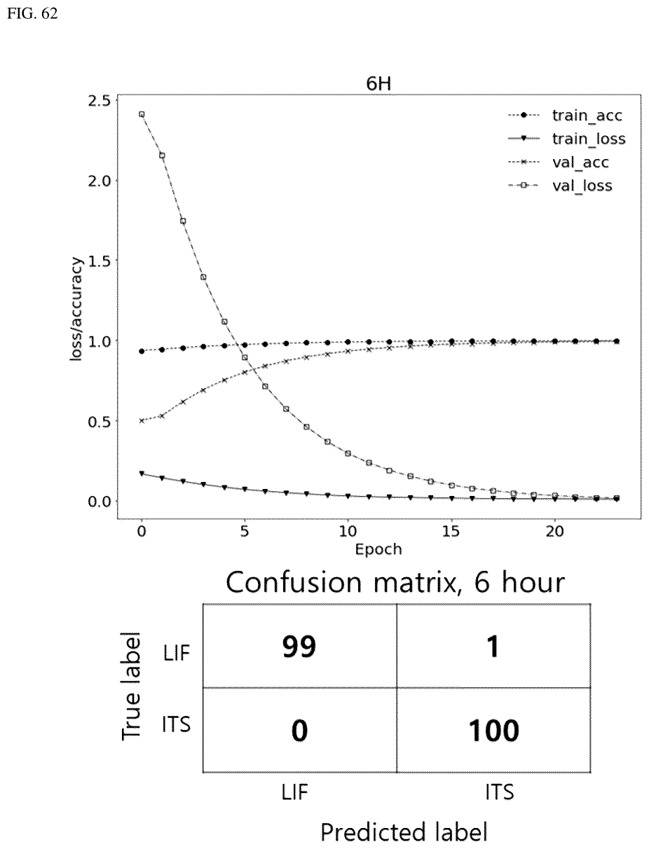

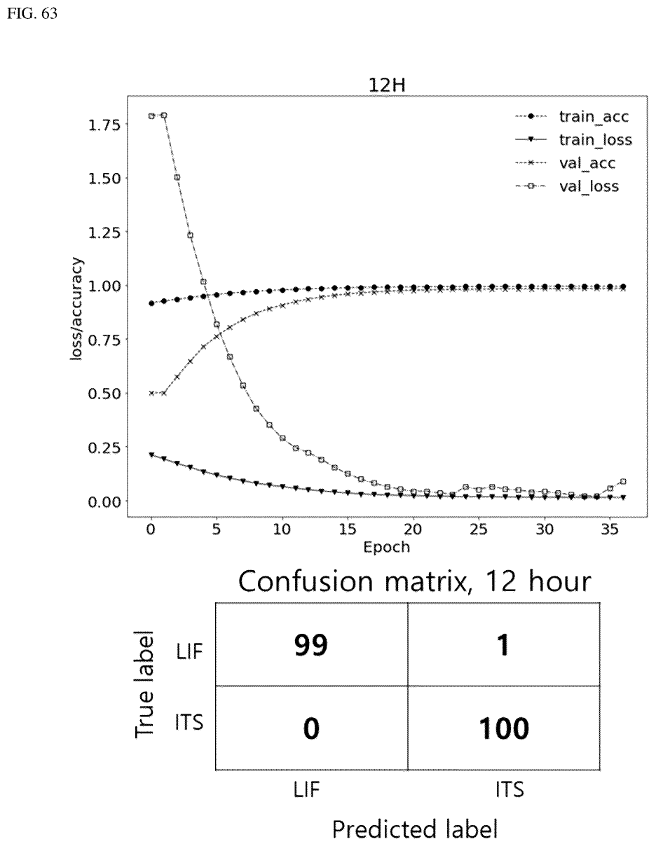

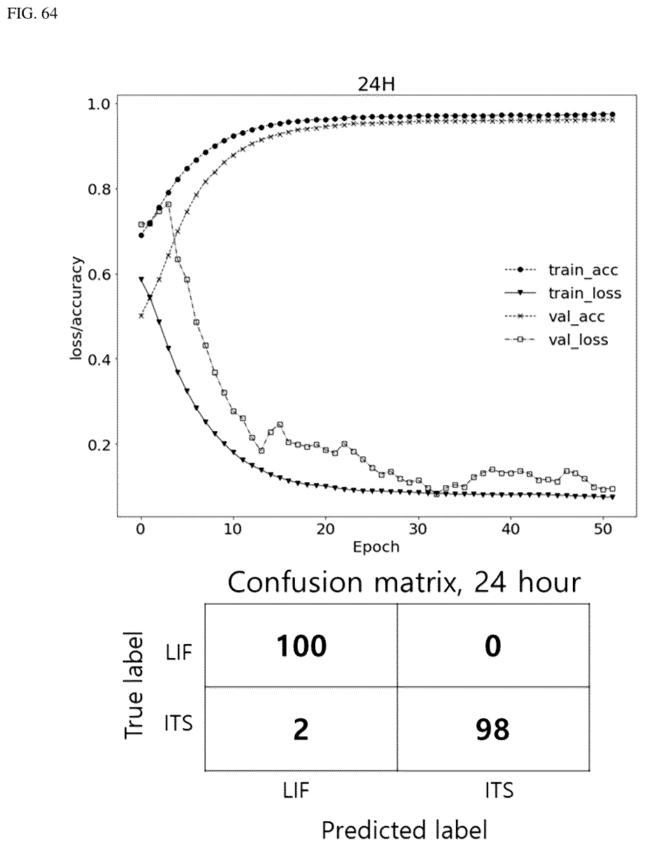

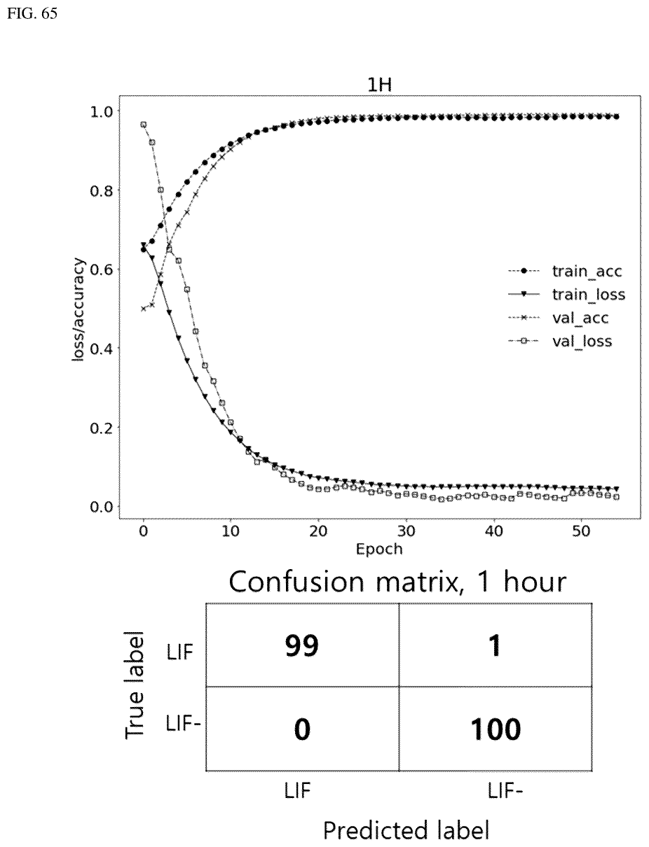

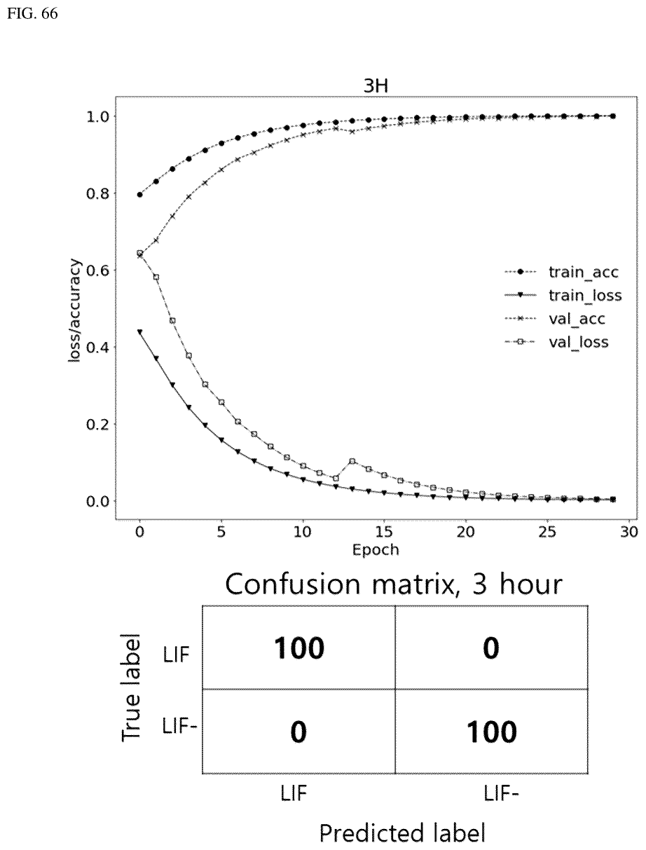

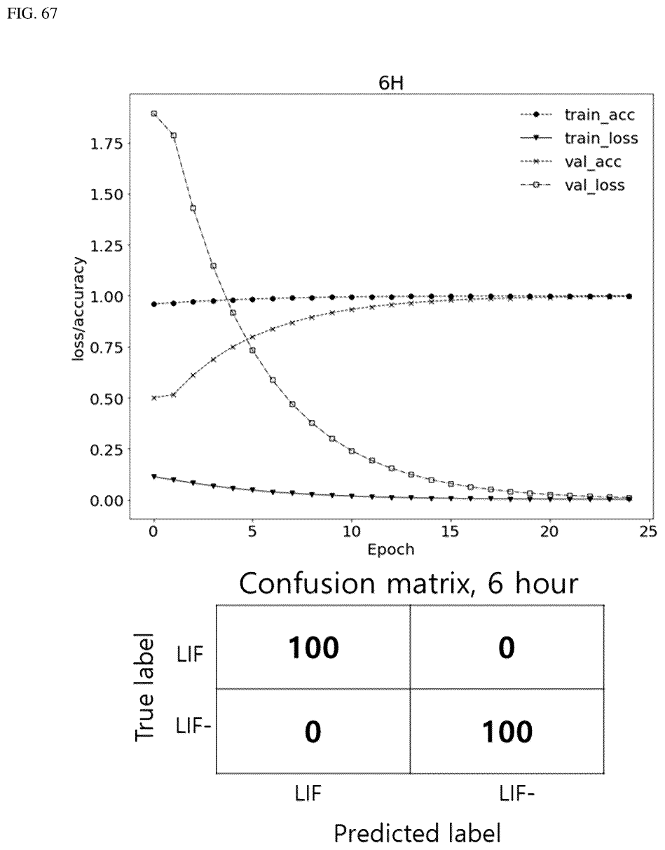

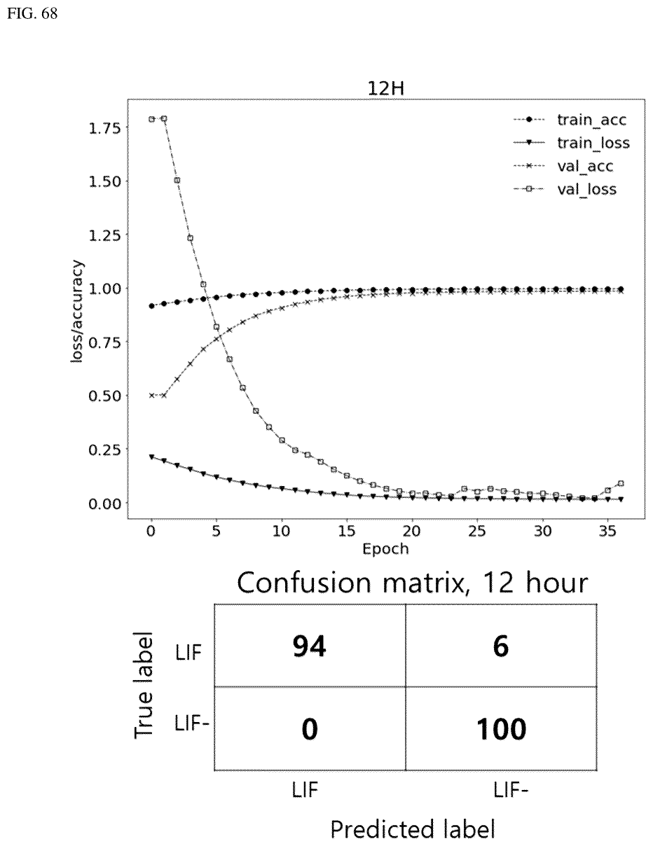

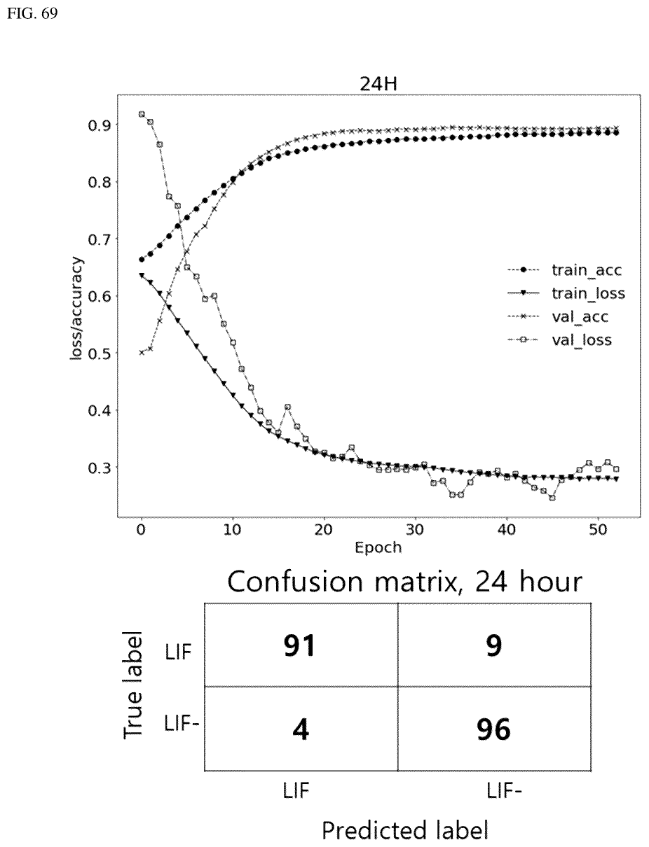

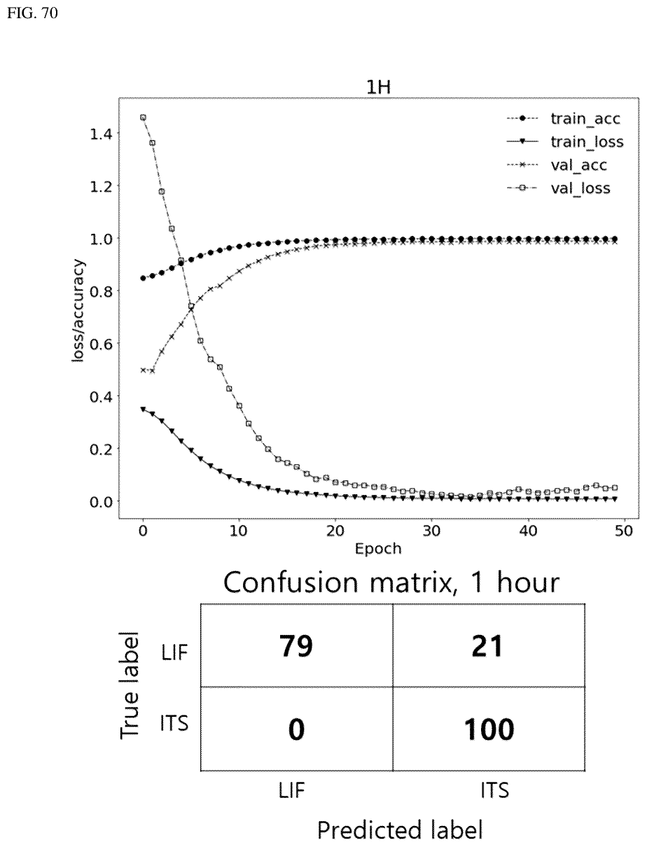

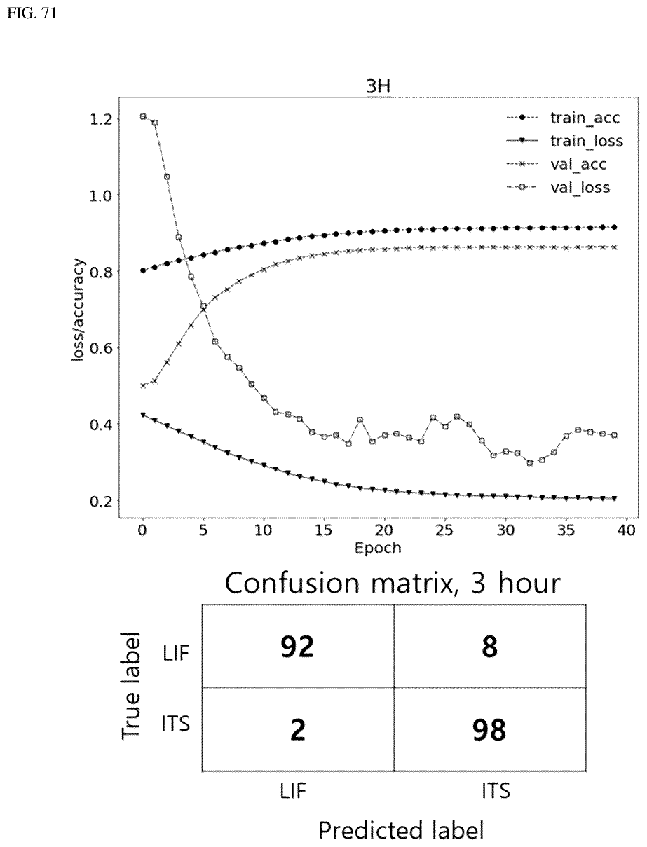

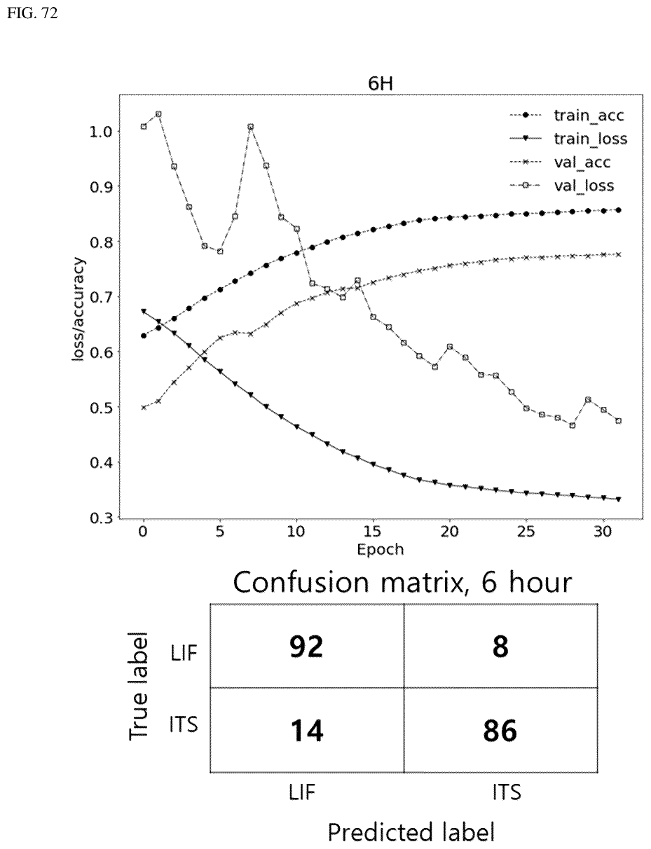

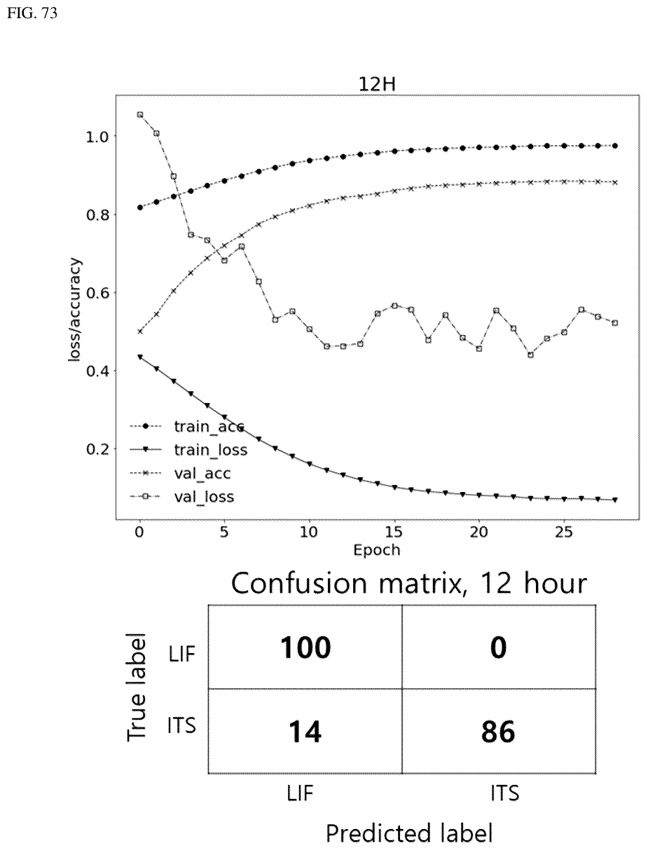

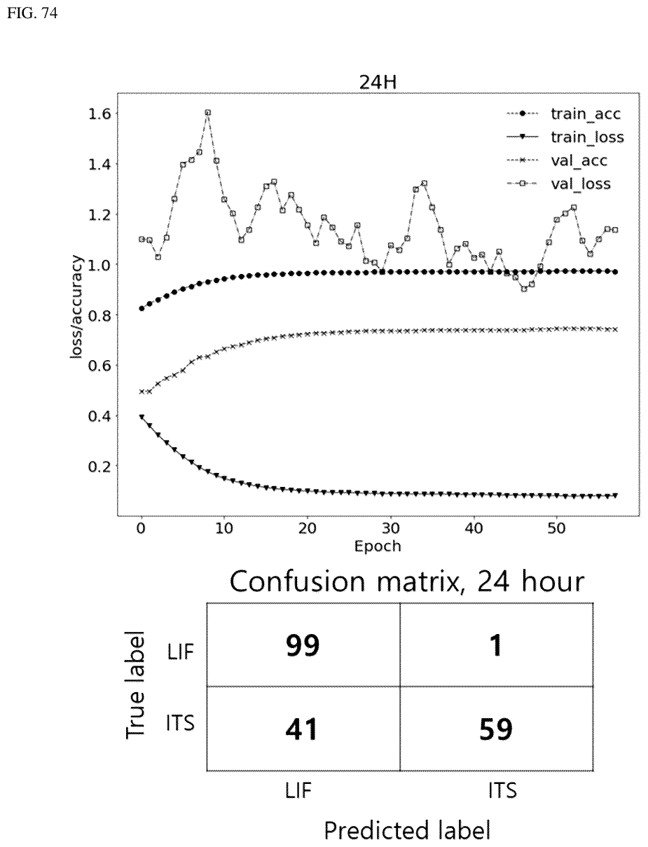

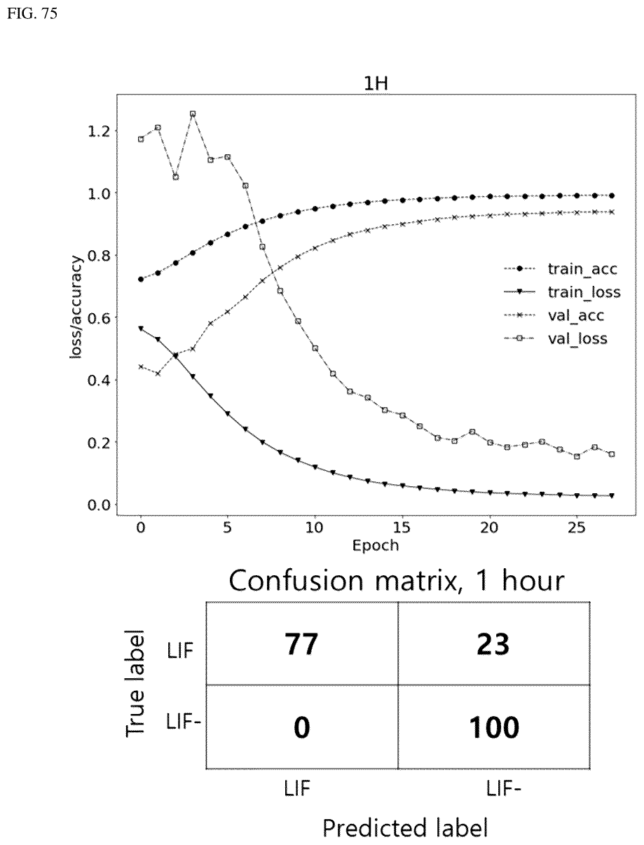

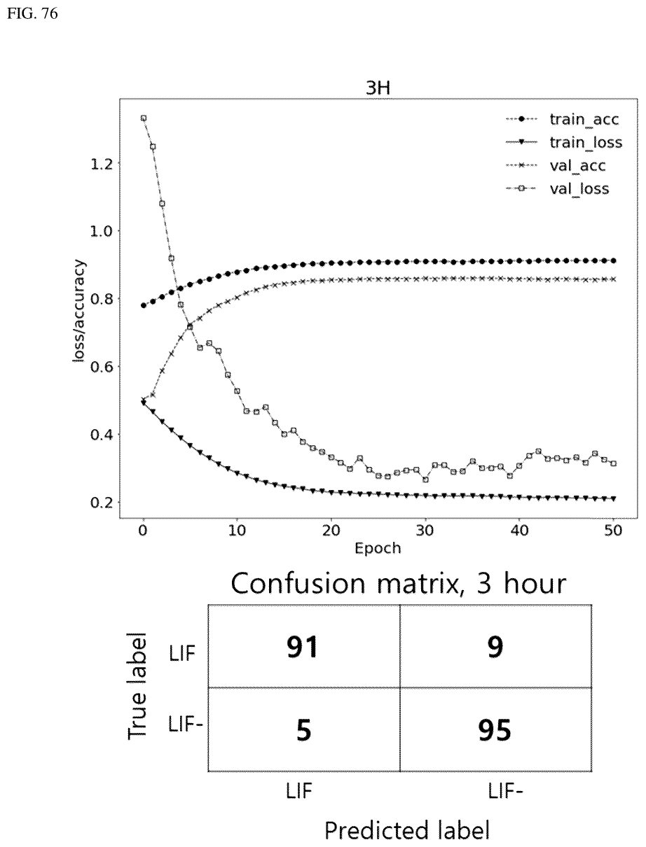

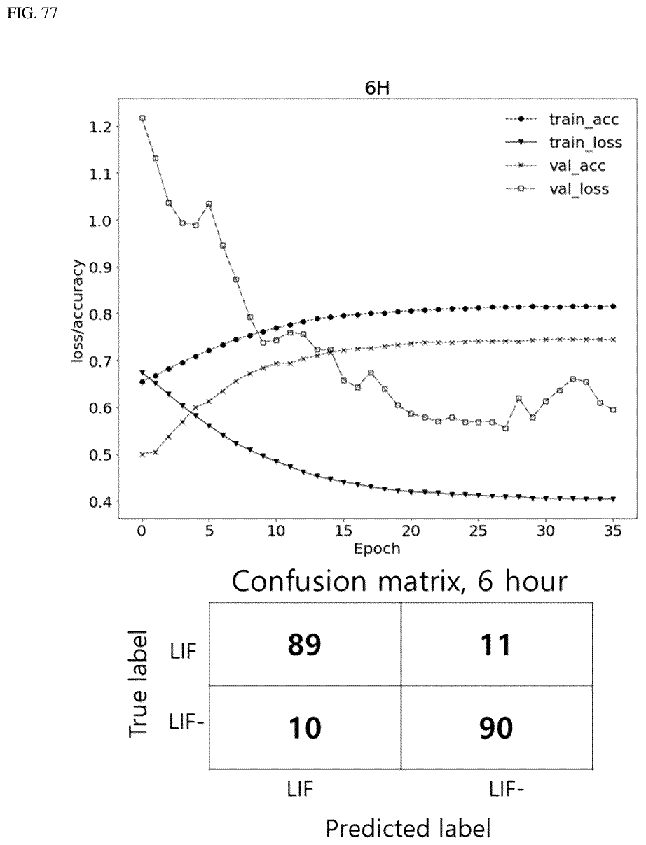

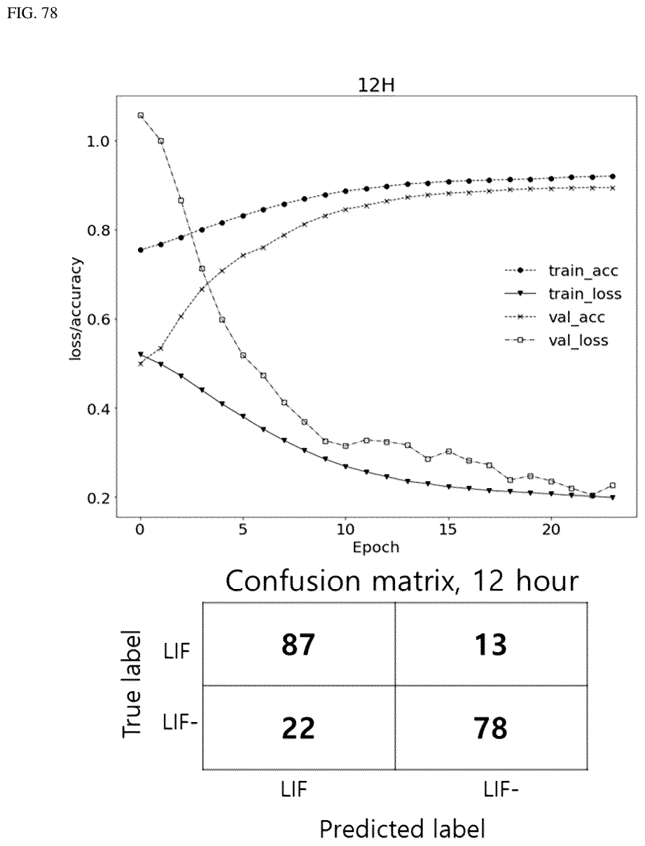

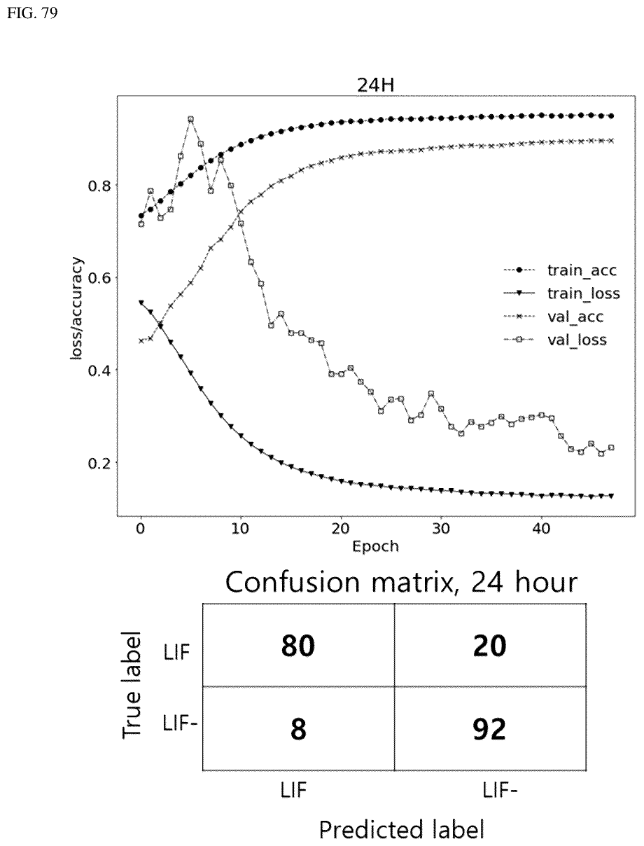

The mouse induced pluripotent stem cells (miPSCs−) were cultured using LIF and ITS media, and using media to which LIF was added and using media from which LIF was removed (marked LIF−). The training results are summarized in FIGS. 70 to 74 and FIGS. 75 to 79.

Referring to FIGS. 70 to 74, in a left graph, the validation loss was generally unstable after the time zone of 6 hours. In the last time zone of 24 hours, the training accuracy was high, while the validation loss was too large because a large amount of cell debris was generated and the cell proliferation rate was low. In the time zone of 1 hour, the accuracy of distinguishing cells cultured in the LIF media was 79%. In the time zone of 24 hours, the accuracy of distinguishing cells cultured in the ITS was as low as 59%. In the time zone of 24 hours, a lot of cell debris was generated and the cell proliferation rate was high such that it was difficult to distinguish the cell shape, resulting in the unstable validation loss and the low test accuracy.

Referring to FIGS. 75 to 79, in an upper graph, the accuracy of each of the training and the validation was generally high, while the validation loss was unstable in the time zone of 24 hours. In a lower confusion matrix, the accuracy was not as high as in the results about the other types of cells.

As described above, as a result of training using the set of 2,000 training images, the overfitting phenomenon occurring in training using the set of 1,500 training images did not occur, and the high accuracy having significancy was obtained in the mouse induced pluripotent stem cells (miPSCs−) for which it was difficult to obtain the high accuracy.

Based on these results, the method and the device of the present disclosure may provide the possibility of distinguishing the cell shape change according to the media type at high accuracy in a future unmanned automated cell culturing system.

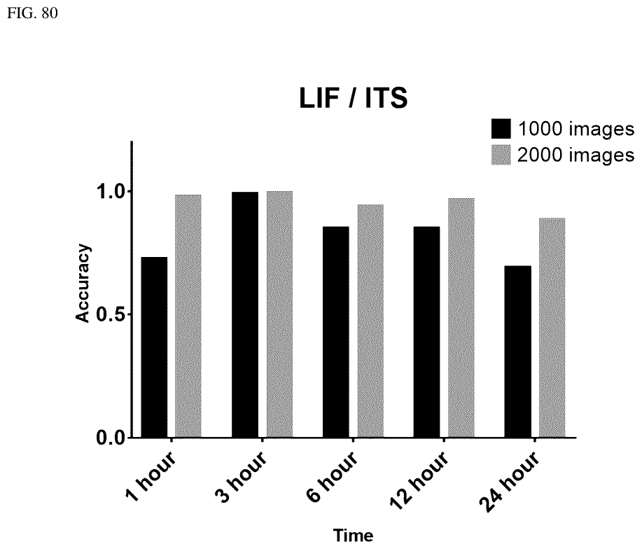

FIG. 80 is a graph showing the results of comparing of the training accuracies of the training sets in the cell discriminating method using artificial intelligence deep learning according to one embodiment of the present disclosure.

Referring to FIG. 80, it may be identified that in terms of accuracy in distinguishing the LIF (leukemia inhibitory factor) media for maintenance purposes and the ITS (insulin-transferrin-selenite) media for differentiation purposes from each other, as the number of training data is greater, that is, when using the set of 2,000 training data is used, the training accuracy is higher, compared to that when using the set of 1,000 training data.

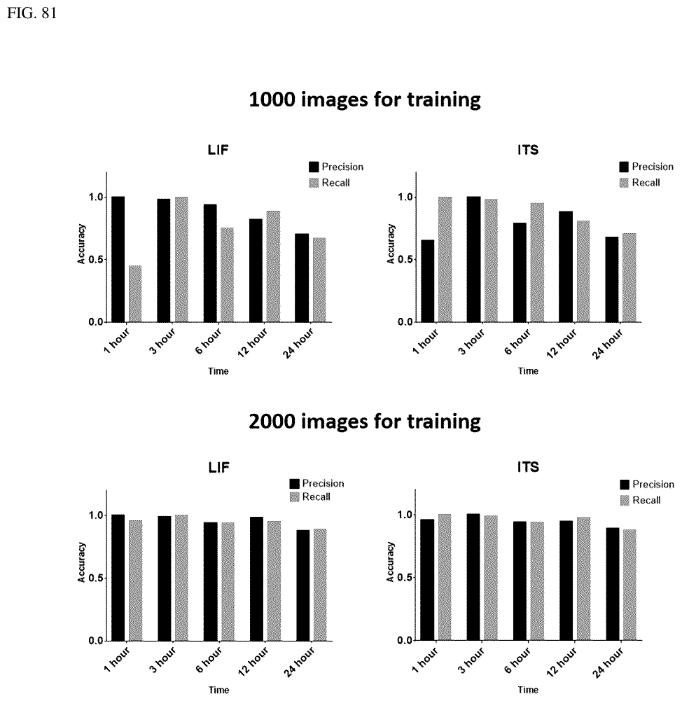

FIGS. 81 to 86 are graphs showing the precision and the recall of cell morphology learning in culturing various types of cells in each of media conditions according to the present disclosure. In this regard, the precision may refer to a ratio of the number of images being actually true among the number of images being classified as true in the model learning. The recall may refer to a ratio of the number of images which the model predicts to be true among the number of images being actually true.

Referring to FIG. 81, in training using a set of 1,000 training images of cells cultured in LIF media (see the left graph in the upper graphs), the precision of cell morphology learning decreases over time. Furthermore, the recall of cell morphology learning was highest in the time zone of 3 hours, while the overall recall value was not high. Likewise, in training using a set of 1,000 training images of cells cultured in ITS media for differentiation purposes(see the right graph in the upper graphs), the precision and the recall were the highest in the time zone of 3 hours, while the overall unstable and low values were recorded.

However, it may be identified that in training using a set of 2,000 training images of cells cultured in each of the LIF media and the ITS media (see the lower graph), both precision and recall of cell morphology learning were high.

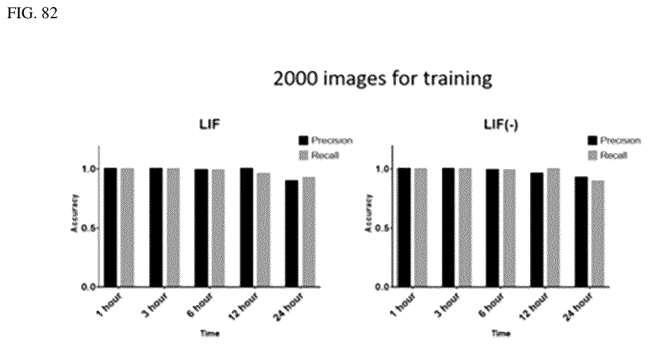

Referring to FIG. 82, in training using a set of 2,000 training images, both the precision and recall values related to the cells cultured in each of the LIF containing media (LIF) and the media free of the LIF (LIF−) were high. Furthermore, in the time zone of 24 hours, the precision was relatively low due to limitation in distinguishing the cell morphology due to cell proliferation, but was high.

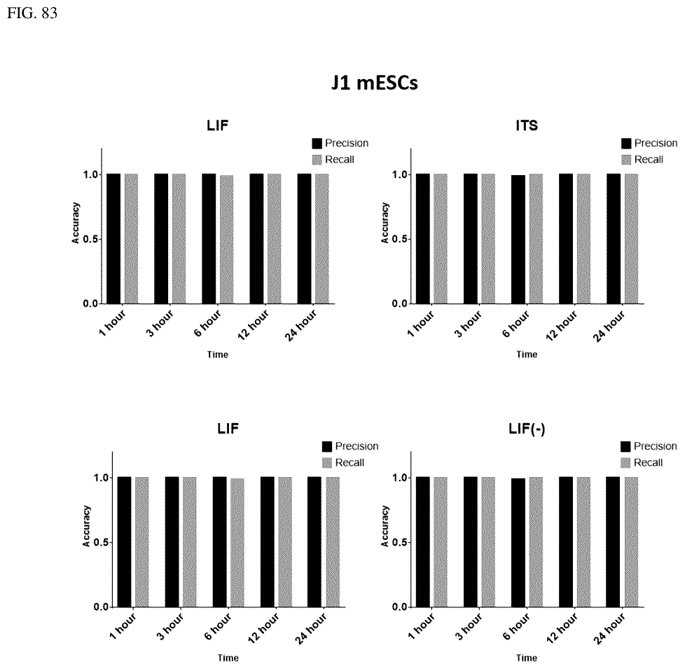

Referring to FIG. 83, the precision and the recall of discrimination of the cells (J1 mESCs (mouse embryonic stem cells) cells) cultured in each media condition based on the shape thereof were high values close to 1 in all time zones and in LIF media and ITS media, LIF media and LIF(−) media. This means that the CNN model can distinguish J1 mESCs cells (mouse embryonic stem cell J1). Therefore, this algorithm may be applied to various types of cells.

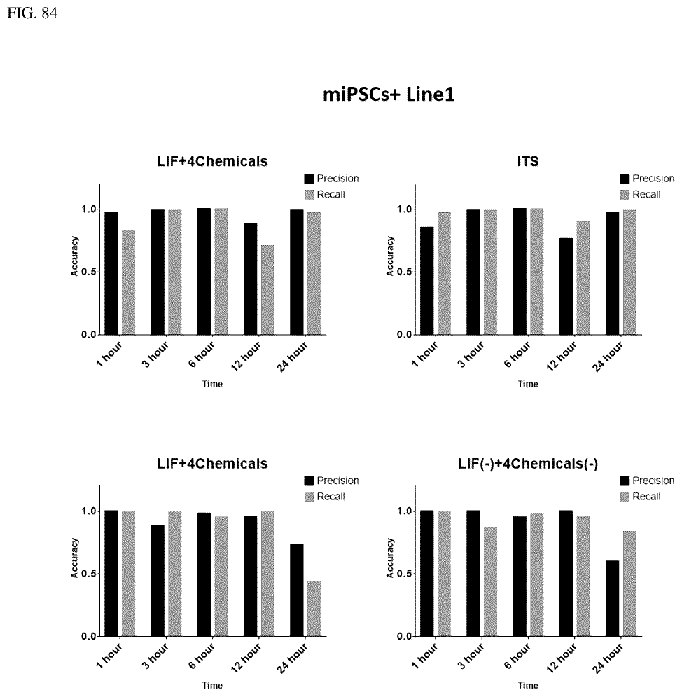

Referring to FIG. 84, the precision and the recall of discrimination of the cells (miPSCs (mouse induced pluripotent stem cells) Line1) cultured in each media condition based on the shape thereof were low due to the generation of cellular debris in the time zone of 12 hours, compared to those in the other time zones, in the LIF+4 chemicals (e.g., containing PD0325901, SB431542, thiazovivin, and ascorbic acid) media and in the ITS media for differentiation (see upper two graphs). Furthermore, regarding the cells cultured in the LIF+4chemicals media and in the media (LIF(−)+4chemicals(−)) from which the LIF and 4chemicals were removed, due to the limitations in imaging only the shape of the cells because of the cell proliferation and the debris generation in the time zone of 24 hours, the precision and the recall were lower in the time zone of 24 hours than those in the other time zones (see lower two graphs). However, the precision and recall were high in the other time zones than the time zone of 24 hours.

Referring to FIG. 85 which shows the precision and the recall of discrimination of the cells (miPSCs (mouse induced pluripotent stem cells) Line2) cultured in each media condition (the same as the condition in FIG. 84) based on the shape thereof, the precision and the recall showed values close to 1 across all time zones.

Therefore, the cell shape has variations depending on the cell passage or the researcher. However, according to the results of training with the CNN model, even when the cell shapes are slightly different from each other, the subtle differences can be distinguished from each other, and consistent results can be produced at high accuracy.

For reference, Line1 and Line2 represent different cell lines as cultivated under the same culturing condition.

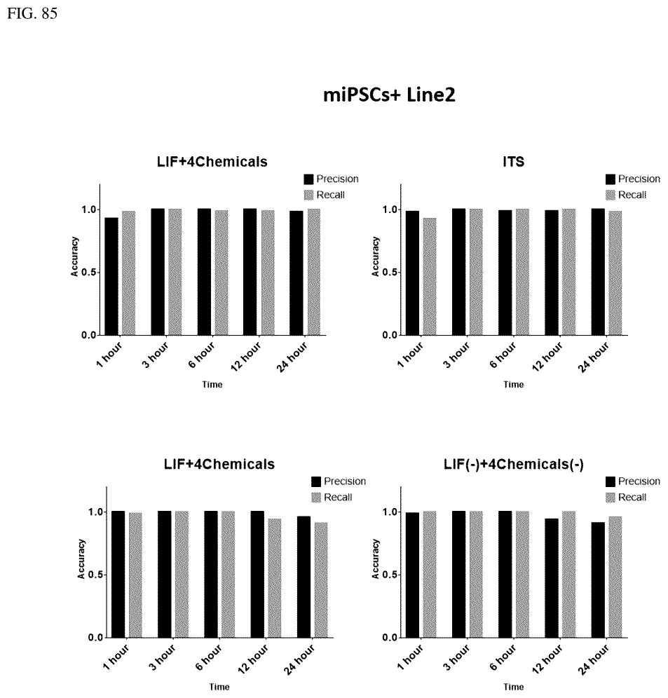

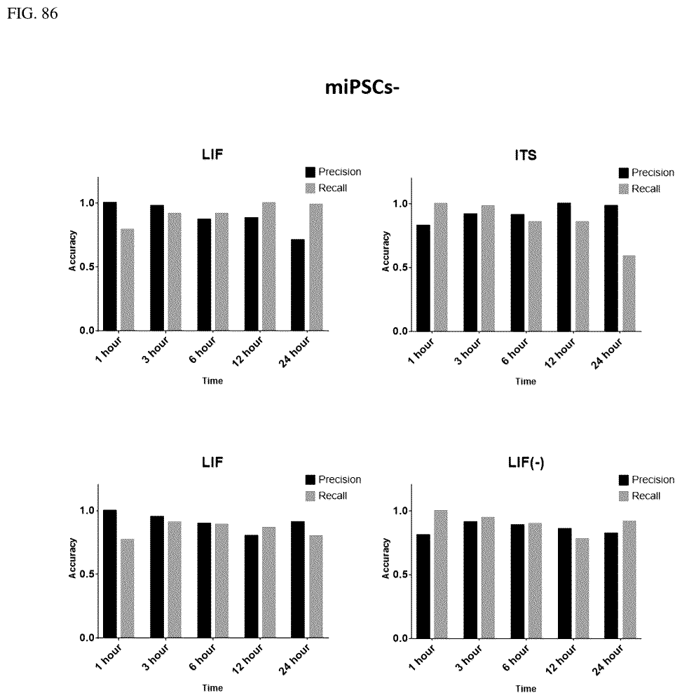

FIG. 86 shows the precision and the recall of discrimination of the cells (miPSCs−) cultured in each media condition (the same as the condition in FIG. 82) based on the shape thereof.

In the media with LIF added thereto, the precision decreased across various time zones, while the recall increased across various time zones (see the left graph of the upper two graphs). In this regard, the training accuracy decreased across various time zones due to the generation of cellular debris. However, the recall as the ratio of the number of the images being actually true among the number of the images being determined to be true by the CNN model regarding the cells cultured in the LIF media increased. Thus, it may be determined that the training was performed properly at a certain level.

Regarding the cells cultured in the ITS media that induces the differentiation, the precision increased across various time zones, while the recall decreased across various time zones (see the graph on the right of the upper two graphs). Because the shape of the cell cultured in the ITS media changed rapidly and a lot of cell debris were generated, the recall was somewhat low even though the training accuracy was high.

Regarding the LIF containing media and the media from which LIF was removed (LIF(−)), the recall of the cells cultured in the LIF containing media was generally high, except for the low value in the time zone of 1 hour. The cell differentiation ability in the two media conditions means that the CNN model is able to distinguish the difference in the cell change well even though it is difficult to visually distinguish the difference in the cell change with the human eyes (see the lower two graphs).

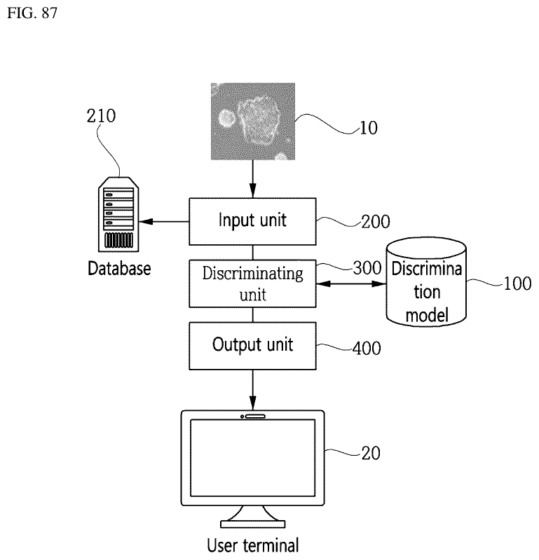

Further, FIG. 87 is a block diagram showing a cell discriminating device using artificial intelligence deep learning according to one embodiment of the present disclosure.

Referring to FIG. 87, the cell discriminating device using artificial intelligence deep learning according to an embodiment of the present disclosure may include an input unit 200 to which the cell image 10 is input, a discriminating unit 300 that uses the deep learning-based discriminating model 100 to discriminate at least one of various cell types, various culturing conditions, and culturing times corresponding to the input cell image 10, and an output unit 400 that provides the discriminating result of the discriminating unit 300 to a user terminal 20.

The cell image 10 input through the input unit 200 may be stored in database 210.

The user terminal 20 may refer to a device used by a user for cell discrimination. In other words, the user terminal 20 may include any device that may provide the result of discriminating the cells based on the cell image 10 to the user through a display or sound signal.

Furthermore, according to one embodiment of the present disclosure, a computer-readable recording medium in which a program for implementing the above-described method is recorded may be provided.

In one example, the above method may be written as a program that may be executed on a computer or may be implemented in a general-purpose digital computer that executes the program using a computer-readable medium. Furthermore, a structure of the data as used in the above-described method may be recorded in computer-readable medium via various means. A recording medium recording therein an executable computer program or code for performing the various steps of the methods of the present disclosure should not be construed as including transient things such as carrier waves or signals. The computer-readable media may include storage media such as magnetic storage media (e.g., ROM, floppy disk, hard disk, etc.) and optical readable media (e.g., CD-ROM, DVD, etc.).

The descriptions of the present disclosure as set forth above are for illustrative purposes. Anyone with ordinary knowledge in the technical field to which the present disclosure belongs may understand that the present disclosure may be easily modified into another specific forms without changing the technical idea or essential features of the present disclosure. Therefore, the embodiments as described above should be understood in all respects as illustrative and not restrictive. For example, each component that is described as a single type component may be implemented in a distributed manner, or components that are described as distributed components may be implemented in a combined manner.

The scope of the present disclosure is indicated by the patent claims as described later rather than the detailed descriptions, and the meaning and scope of the patent claims and all changes or modified forms derived from the equivalent concept are interpreted as being included in the scope of the present disclosure.

What is claimed is:

1. A cell discrimination method using artificial intelligence, the method comprising:
an input step of inputting a cell image; and
a discriminating step of discriminating at least one of various cell types, various culturing conditions, and various culturing times corresponding to the input cell image using a deep learning-based discriminating model,
wherein the discriminating step includes:
extracting a first feature in the cell image;
extracting a second feature in the cell image; and
determining the at least one of the cell types, the culturing conditions, and the culturing times corresponding to the cell image, based on the extracted first feature and second feature,
wherein the discriminating model includes:
a first neural network configured to extract the first feature from the cell image;
a second neural network configured to extract the second feature from the cell image; and
a fully connected layer configured to determine the at least one of the cell types, the culturing conditions, and the culturing times corresponding to the input cell image, based on the extracted first feature and second feature.

2. The cell discrimination method of claim 1, wherein the cell image is captured in at least one of following time zones: 1 hour to 1 hour and 30 minutes, 3 hours to 3 hours and 30minutes, 6 hours to 6 hours and 30 minutes, 12 hours to 12 hours and 30 minutes, and 24 hours to 24 hours and 30 minutes after the cell culturing.

3. The cell discrimination method of claim 1, wherein the first neural network is implemented as a shallow-structured convolution neural network composed of one convolution layer and one pooling layer,
wherein the second neural network is implemented as a deep-structured convolution neural network composed of four convolution layers.

4. The cell discrimination method of claim 1, wherein the various cell types include animal cells and human cells including at least one of stem cell lines, human skin fibroblast cell lines, epithelial cell lines, and immune cell lines,
wherein the various culturing conditions are different from each other for each cell type.

5. The cell discrimination method of claim 4, wherein the stem cell line includes at least one of mouse embryonic stem cell, mouse induced pluripotent stem cell, human embryonic stem cell, human induced pluripotent stem cell, human neural stem cell, human hair follicle stem cell, human mesenchymal stem cell, and human fibroblast cell,
wherein the epithelial cell line includes human skin keratinocyte (HaCaT),
wherein the immune cell line includes a T cell,
wherein the human neural stem cells includes a human somatic cell-derived cell converted neural stem cell or a human brain-derived neural stem cell.

6. The cell discrimination method of claim 5, wherein the culturing condition for the mouse embryonic stem cell includes at least one of:
a culturing condition including LIF (leukaemia inhibitory factor) media;
a culturing condition including ITS (insulin-transferrin-selenium supplement) media; and
a culturing condition excluding the LIF media,
wherein the culturing condition for the mouse induced pluripotent stem cell includes at least one of:
a culturing condition including PD0325901, SB431542, thiazovivin, ascorbic acid, and LIF media;
a culturing condition excluding PD0325901, SB431542, thiazovivin, ascorbic acid, and LIF media; and
a culturing condition including ITS media,
wherein the culturing condition for the human embryonic stem cell or the human induced pluripotent stem cell includes at least one of:
a culturing condition including PD0325901, SB431542, thiazovivin, ascorbic acid, and LIF media;
a culturing condition excluding PD0325901, SB431542, thiazovivin, ascorbic acid, and LIF media; and a culturing condition including ITS media,
wherein the culturing condition for the human somatic cell-derived cell-converted neural stem cell includes at least one of:
a culturing condition including DMEM/F12, N2, B27, bFGFF, EGF, thiazovivin, valproic acid, Purmorphamine, A8301, SB431542, CHIR99021, Deazaneplanocin A (DZNep), and Azacitidine (5-AZA);
a culturing condition including DMEM/F12, N2, B27, bFGF, and EGF; and
a culturing condition including DMEM/F12 and ITS media,
wherein the culturing condition for the human brain-derived neural stem cell includes at least one of:
a culturing condition including a basic medium, an induced neural stem cell growth supplement and antibiotics;
a culturing condition including the basic medium and antibiotics; and
a culturing condition including the basic medium, antibiotics, and ITS media,
wherein the culturing condition for the human hair follicle stem cells includes at least one of:
a culturing condition in which 10% FBS (Fetal bovine serum), Pen/Strep (Penicillin & Streptomycin), L-glutamine, and streptomycin are contained in DMEM media; and
a culturing condition in which ITS media is contained in DMEM media,
wherein the culturing condition for the human mesenchymal stem cells includes at least one of:
a culturing condition in which 10% FBS (Fetal bovine serum), NEAA (non-Essential Amino Acids), and Pen/Strep are contained in DMEM media; and
a culturing condition in which the ITS media is contained in DMEM media,
wherein the culturing condition for the human fibroblast includes a culturing condition in which 10% FBS, Pen/Strep, and NEAA are contained in DMEM media,
wherein the culturing condition for the HaCaT cells includes a culturing condition in which 10% FBS, Pen/Strep, L-glutamine, and streptomycin are contained in DMEM media.

7. The cell discrimination method of claim 1, wherein the discriminating model includes a data set for image learning, wherein the data set includes each of a set of 1,000 training images, a set of 1,500 training images, and a set of 2,000 training images, and a set of 800 validation images, and a set of 100 test images.

8. The cell discrimination method of claim 6, wherein the discriminating model is configured to adopt a set of 2,000 training images.

9. A cell discriminating device using artificial intelligence, the device comprising:
an input unit for receiving a cell image;
a discriminating unit configured to discriminate at least one of various cell types, various culturing conditions, and various culturing times corresponding to the cell image using a deep learning-based discriminating model; and
an output unit configured to provide the discriminating result of the discriminating unit to a user terminal,
wherein the discriminating unit is configured to:
extract a first feature in the cell image;
extract a second feature in the cell image; and
determine the at least one of the cell types, the culturing conditions, and the culturing times corresponding to the cell image, based on the extracted first feature and second feature,
wherein the discriminating model includes:
a first neural network configured to extract the first feature from the cell image;
a second neural network configured to extract the second feature from the cell image; and
a fully connected layer configured to determine the at least one of the cell types, the culturing conditions, and the culturing times corresponding to the input cell image, based on the extracted first feature and second feature.

10. The cell discrimination device of claim 9, wherein the cell image is captured in at least one of following time zones: 1 hour to 1 hour and 30 minutes, 3 hours to 3 hours and 30 minutes, 6 hours to 6 hours and 30 minutes, 12 hours to 12 hours and 30 minutes, and 24 hours to 24 hours and 30 minutes after the cell culturing.

11. The cell discrimination device of claim 9, wherein the first neural network is implemented as a shallow-structured convolution neural network composed of one convolution layer and one pooling layer,
wherein the second neural network is implemented as a deep-structured convolution neural network composed of four convolution layers.

12. The cell discrimination device of claim 9, wherein the various cell types include animal cells and human cells including at least one of stem cell lines, human skin fibroblast cell lines, epithelial cell lines, and immune cell lines,
wherein the various culturing conditions are different from each other for each cell type.

13. The cell discrimination method of claim 12, wherein the stem cell line includes at least one of mouse embryonic stem cell, mouse induced pluripotent stem cell, human embryonic stem cell, human induced pluripotent stem cell, human neural stem cell, human hair follicle stem cell, human mesenchymal stem cell, and human fibroblast cell,
wherein the epithelial cell line includes human skin keratinocyte (HaCaT),
wherein the immune cell line includes a T cell,
wherein the human neural stem cells includes a human somatic cell-derived cell converted neural stem cell or a human brain-derived neural stem cell.

14. The cell discrimination method of claim 13, wherein the culturing condition for the mouse embryonic stem cell includes at least one of:
a culturing condition including LIF (leukaemia inhibitory factor) media;
a culturing condition including ITS (insulin-transferrin-selenium supplement) media; and
a culturing condition excluding the LIF media,
wherein the culturing condition for the mouse induced pluripotent stem cell includes at least one of:
a culturing condition including PD0325901, SB431542, thiazovivin, ascorbic acid, and LIF media;
a culturing condition excluding PD0325901, SB431542, thiazovivin, ascorbic acid, and LIF media; and
a culturing condition including ITS media,
wherein the culturing condition for the human embryonic stem cell or the human induced pluripotent stem cell includes at least one of:
a culturing condition including PD0325901, SB431542, thiazovivin, ascorbic acid, and LIF media;
a culturing condition excluding PD0325901, SB431542, thiazovivin, ascorbic acid, and LIF media; and a culturing condition including ITS media,
wherein the culturing condition for the human somatic cell-derived cell-converted neural stem cell includes at least one of:
a culturing condition including DMEM/F12, N2, B27, bFGFF, EGF, thiazovivin, valproic acid, Purmorphamine, A8301, SB431542, CHIR99021, Deazaneplanocin A (DZNep), and Azacitidine (5-AZA);
a culturing condition including DMEM/F12, N2, B27, bFGF, and EGF; and
a culturing condition including DMEM/F12 and ITS media,
wherein the culturing condition for the human brain-derived neural stem cell includes at least one of:
a culturing condition including a basic medium, an induced neural stem cell growth supplement and antibiotics;
a culturing condition including the basic medium and antibiotics; and
a culturing condition including the basic medium, antibiotics, and ITS media,
wherein the culturing condition for the human hair follicle stem cells includes at least one of:
a culturing condition in which 10% FBS (Fetal bovine serum), Pen/Strep (Penicillin & Streptomycin), L-glutamine, and streptomycin are contained in DMEM media; and
a culturing condition in which ITS media is contained in DMEM media,
wherein the culturing condition for the human mesenchymal stem cells includes at least one of:
a culturing condition in which 10% FBS (Fetal bovine serum), NEAA (non-Essential Amino Acids), and Pen/Strep are contained in DMEM media; and
a culturing condition in which the ITS media is contained in DMEM media,
wherein the culturing condition for the human fibroblast includes a culturing condition in which 10% FBS, Pen/Strep, and NEAA are contained in DMEM media,
wherein the culturing condition for the HaCaT cells includes a culturing condition in which 10% FBS, Pen/Strep, L-glutamine, and streptomycin are contained in DMEM media.

15. The cell discrimination device of claim 9, wherein the discriminating model includes a data set for image learning, wherein the data set includes each of a set of 1,000 training images, a set of 1,500 training images, and a set of 2,000 training images, and a set of 800 validation images, and a set of 100 test images.

16. The cell discrimination method of claim 15, wherein the discriminating model is configured to adopt a set of 2,000 training images.
